(19) United States
(12) Patent Application Publication
(10) Pub. No.: US 2024/0366571 A1
(43) Pub. Date: Nov. 7, 2024

(54) MODULATORS OF SK4 POTASSIUM CHANNEL AND USES THEREOF

(71) Applicants: Ramot at Tel-Aviv University Ltd., Tel-Aviv (IL); B.G. Negev Technologies and Applications Ltd., at Ben-Gurion University, Beer-Sheva (IL)

(72) Inventors: Bernard ATTALI, Tel-Aviv (IL); Shahaf Asher PERETZ, Tel-Aviv (IL); Elvira HAIMOV, Tel-Aviv (IL); Boris REDKO, Tel-Aviv (IL); Avi RAVEH, Tel-Aviv (IL); Adva YEHESKEL, Tel-Aviv (IL); Hamutal ENGEL, Tel-Aviv (IL); Yoram ETZION, LeHavim (IL)

(73) Assignees: Ramot at Tel-Aviv University Ltd., Tel-Aviv (IL); B.G. Negev Technologies and Applications Ltd., at Ben-Gurion University, Beer-Sheva (IL)

(21) Appl. No.: 18/775,105

(22) Filed: Jul. 17, 2024

Related U.S. Application Data
(63) Continuation of application No. PCT/IL2023/050048, filed on Jan. 17, 2023.
(60) Provisional application No. 63/300,070, filed on Jan. 17, 2022.

(51) Int. Cl. A61K 31/4184; A61P 9/06

(52) U.S. Cl. CPC A61K 31/4184; A61P 9/06

(57) ABSTRACT

Compounds capable of interacting with the calmodulin-PIP2 binding domain of the SK4 channel to thereby interfere with the Ca²⁺-dependent activation of the SK4 channel and uses thereof in downregulating an activity of an SK4 channel in a subject in need thereof, are disclosed. Also disclosed is a method of identifying a candidate compound that is capable of downregulating an activity of SK4 channel, based on its interaction with the calmodulin-PIP2 binding domain of the SK4 channel.

### RELATED APPLICATIONS

This application is a Continuation of PCT Patent Application No. PCT/IL2023/050048 having International filing date of Jan. 17, 2023, which claims the benefit of priority under 35 USC § 119(e) of U.S. Provisional Patent Application No. 63/300,070 filed on Jan. 17, 2022. The contents of the above applications are all incorporated by reference as if fully set forth herein in their entirety.

### FIELD AND BACKGROUND OF THE INVENTION

The present invention, in some embodiments thereof, relates to therapy and, more particularly, but not exclusively, to novel compounds that modulate (e.g., downregulate) an activity of Ca²⁺-activated potassium channel SK4, which are usable in the treatment of arrhythmic disorders and other medical conditions that are associated with SK4 activity and/or in which downregulating an activity of SK4 is beneficial.

The potassium channel family, which is activated by intracellular Ca²⁺, consists of four members based on their single channel conductance: small-conductance (SK) channels (5-10 pS) comprising three members (SK1-SK3), and an intermediate conductance (IK) SK4 channel (20-80 pS). SK4 K⁺ channels are encoded by the gene KCNN4 and share the same tetrameric architecture as voltage-gated K⁺ channels, where each subunit is endowed with six transmembrane helices (S1-S6), and cytoplasmic N- and C-termini. Although exhibiting similar design as the small-conductance potassium channels, SK4 K⁺ channels are not activated by depolarization, but are gated by Ca²⁺-bound calmodulin (CaM). CaM is tethered to a CaM-binding domain (CaMBD) located at the proximal C-terminus of the channel, and contacting the S4-S5 intracellular linker. A recent cryo-EM structure of the human SK4 K⁺ channel [PDB code: 6CNN; in Lee et al., Science (2018) 360, 508] showed four CaM molecules per channel tetramer. It revealed that the CaM C-lobe interacts with the proximal C-terminus in a Ca²⁺-independent manner. The calcified form of CaM N-lobe interacts with the S4-S5 linker to sense Ca²⁺ and gate the channel.

SK4 K⁺ channels are expressed in the immune system, in T cells, B cells, mast cells, macrophages, microglia, and others. In immune cells, these channels hyperpolarize the cell membrane, which drives calcium ions entry and necessitating them for activation, proliferation, and production of cytokines [Cahalan et al., Immunol Rev (2009) 231, 59-87; Feske et al., Annu Rev Immunol (2015) 33, 291-353; Kaushal et al., J Neurosci (2007) 27, 234-244; Nguyen et al., Glia (2017) 65, 106-121; Shumilina et al., J Immunol (2008) 180, 8040-8047; Wulff et al., J Immunol (2004) 173, 776-786].

SK4 K⁺ channels are also expressed in restricted areas of the brain such as the hippocampus and cerebellum, where they contribute to the slow after-hyperpolarization [Engbers et al., Proc Natl Acad Sci USA (2012) 109, 2601-2606; King et al., Cell Rep (2015) 11, 175-182].

Expression of SK4 channels in various cancerous processes has also been demonstrated [see, for example, Zhang et al., PLoS One (2016) 11, e0154471; Yang et al., J Hua Zhong U Sci Technol Med Sci (2013) 33, 86-89; McFerrin et al., Am J Physiol Cell Physiol (2012) 303, C1070-C1078; Ruggieri et al. PLoS One (2012) 7, e47825; Roach et al., Respir Res (2014) 15, 155; Lai et al., Med Oncol (2013) 30, 566; Fu et al. PLoS One (2014) 9, e87410; Brown et al., Curr Neuropharmacol (2018) 16, 618-626]. For example, it has been shown that employing SK4 specific SiRNA and blockers in triple-negative breast cancer (TNBC) cells suppresses TNBC cells proliferation, migration, and epithelial-mesenchymal transition (EMT), suggesting that these channels may act also as a therapeutic target for cancer.

Some of the present inventors have previously identified SK4 K⁺ channels in the mouse sinoatrial node (SAN), and showed its involvement in the pacemaker activity of cardiomyocytes derived from human embryonic stem cells [Weisbrod et al., Proc Natl Acad Sci USA (2013) 110, 18, E1685-94; Weisbrod et al., Acta Pharmacol Sin (2016) 37, 1, 82-97]. Blocking SK4 K⁺ channels reduced the occurrence of delayed-after-depolarization and abnormal Ca²⁺ transients following β-adrenergic receptor stimulation in SAN cells from a mouse model of catecholaminergic polymorphic ventricular tachycardia (CPVT) [Haron-Khun et al., EMBO Mol Med (2017) 4, 415-429].

Further studies have showed that SK4 K⁺ channel blockage produces in-vivo bradycardia, accompanied, in ECG recording, with PR prolongation and reduction of arrhythmic features in CPVT mice model [U.S. Patent Application Publication No. 2018/0177764; Attali et al., Biophys J (2017) 112, 3, 35A]. These studies were conducted while using the currently known SK4 inhibitors clotrimazole and Tram-34.

Inhibition of SK4 K⁺ channels using Tram-34 suppresses in-vivo the vulnerability to atrial fibrillation (AF) in canine heart, suggesting it is likely that additional animal models exhibit similar behaviors when introduced to such inhibitors [Yang et al., Heliyon (2020) 6, 5, e03928; Yang et al., Interv Card Electrophysiol (2021) 60, 2, 247-253].

Current SK4 channel inhibitors focus mainly on the channel pore. The prototypical blockers, such as Tram-34 or clotrimazole, exhibit poor bioavailability and liver toxicity due to interactions with cytochrome P450 enzymes [Wulff et al., Proc Natl Acad Sci USA (2000) 97, 8151-8156; Wulff et al., J Biol Chem (2001) 276, 32040-32045], and were found unsuitable for further clinical trials.

Atrial fibrillation (AF) is the most common sustained cardiac arrhythmia, which affects more than 4% of the population worldwide. Atrial fibrillation is associated with significant mortality, due to embolic stroke and prevalence within ageing population. An estimated 30 million North Americans and Europeans that are expected to suffer from AF by 2050. Currently available drugs or surgery therapy for AF have major limitations including partial efficacy, high recurrence rates, and risk of life-threatening ventricular proarrhythmic side effects.

Other than the known SK4 channel inhibitors, several known benzimidazoles such as 1-ethylbenzimidazolinone (1-EBIO) and other analogous compounds were reported to activate SK channels by binding to the interface between the CaM N-lobe and the S₄₅A linker helix [see examples in Zhang et al., Nat Commun (2012) 3, 1021; and Zhang et al., Sci Adv (2015) 1, e1500008].

Some 1-EBIO analogues were found to exhibit herbicidal and/or fungicidal activities [Giyasov et al., E3S Web of Conferences (2021) 258, 04017].

Additional background art includes Zhang et al., Nat Chem Biol (2014) 10, 753-759; Devor et al., Am J Physiol (1996) 271, L775-784; Joiner et al., Proc Natl Acad Sci USA (1997) 94, 11013-11018; Meyong et al. J Gen Physiol (2020) 152; and Allen et al., J Neurosci (2007) 27, 2369-2376.

### SUMMARY OF THE INVENTION

The Ca²⁺-activated SK4 K⁺ channel is gated by Ca²⁺-calmodulin (CaM) and expressed in immune cells but also in heart. Recent studies suggested that SK4 channel blockers may represent an interesting therapeutic approach for the treatment of cardiac arrhythmias.

Up to date, the pharmacological toolbox of SK4 channel inhibitors has mainly focused on the channel pore. The prototypical blockers, such as Tram-34 or clotrimazole, are not appropriate for clinical development because of poor bioavailability and liver toxicity due to interactions with cytochrome P450 enzymes. Therefore, there is a need to enrich the therapeutic arsenal for treating cardiac arrhythmias more safely and effectively.

The present inventors have identified a previously untargeted region of SK4 channels, the calmodulin (CaM)-PIP2 binding domain (CPBD), at the interface of the proximal C-terminus and the linker S4-S5, and have designed and synthesized novel compounds that act as allosteric SK4 blockers by interfering with the CPBD.

Computational docking studies, while using the human SK4 K+ channel structure, along with patch-clamp electrophysiological validation, revealed that the newly designed compounds inhibit the channel by interacting with two specific residues, R191 and H192, that are not conserved in SK1-SK3 subunits, thereby conferring SK4 selectivity and preventing the calcified CaM N-lobe to correctly contact its S4-S5 linker site to open the channel. The present inventors have also demonstrated that an exemplary compound significantly prolongs atrial and atrioventricular effective refractory periods in rat and guinea-pig isolated hearts and reduces atrial fibrillation (AF) induced by carbachol, further confirming that targeting the CPBD of SK4 K+ channels by allosteric inhibitors offers a novel cardiac anti-AF therapy.

Embodiments of the present invention therefore relate to newly designed compounds, and to downregulating SK4 activity by targeting the CPBD of an SK4 channels using, for example, the newly designed compounds.

According to an aspect of some embodiments of the present invention there is provided a compound for use in downregulating an activity of an SK4 channel in a subject in need thereof, as described herein.

According to an aspect of some embodiments of the present invention there is provided a compound for use in downregulating an activity of an SK4 channel in a subject in need thereof, the compound being capable of interacting with the calmodulin-PIP2 binding domain of the SK4 channel to thereby interfere with the Ca²⁺-dependent activation of the SK4 channel.

According to some of any of the embodiments described herein, the compound is capable of interfering with an interaction of a calcified calmodulin N-lobe with a proximal S₄₅A helix of the SK4 channel.

According to some of any of the embodiments described herein, interfering with the interaction allosterically affects the Ca²⁺-dependent activation of the SK4 channel.

According to some of any of the embodiments described herein, the compound comprises at least one functional moiety that is capable of interacting with at least one amino acid residue at a boundary of the SK4 channel proximal C-terminus and the S4-S5 linker (the PIP2 binding pocket).

According to some of any of the embodiments described herein, the amino acid residue is selected from Arg191 and His192.

According to some of any of the embodiments described herein, the compound comprises at least two functional moieties spatially arranged such that the compound is capable of forming hydrogen bonds and/or π-π stacking interactions with at least two amino acid residues at the boundary.

According to some of any of the embodiments described herein, the at least two amino acid residues comprise Arg191 and His192.

According to some of any of the embodiments described herein, the at least one or at least two functional moieties comprise at least one or at least two functional moieties that feature a hydrogen bond acceptor atom or moiety.

According to some of any of the embodiments described herein, the at least two functional moieties are spatially arranged such that the compound is capable of forming hydrogen bonds with Arg191 and His192.

According to some of any of the embodiments described herein, the compound comprises at least one functional moiety that is capable of interfering with an interaction between at least one amino acid residue of the calmodulin N-lobe that interacts with the linker S4-S5 of the SK4 channel.

According to some of any of the embodiments described herein, the at least one amino acid residue is selected from Met72 and Met76 of calmodulin.

According to some of any of the embodiments described herein, the at least one functional moiety is spatially arranged such that the compound is capable of sterically hinder the at least one amino acid residue (e.g., Met76) of calmodulin, thereby interfering with an interaction of the calmodulin N-lobe with the linker S4-S5 of the SK4 channel.

According to some of any of the embodiments described herein, the at least one functional moiety is spatially arranged such that the compound is capable of forming hydrophobic interactions and/or hydrogen bond interactions with Met72 of calmodulin.

According to some of any of the embodiments described herein, the compound comprises at least two functional moieties that are capable of forming hydrogen bonds and/or π-π stacking interactions, the compound being such that when it interacts with the SK4 channel, it is spatially arranged such that the at least two functional moieties that are capable of forming hydrogen bonds and/or π-π stacking interactions are in proximity and orientation that enable formation of hydrogen bonds and/or π-π stacking interactions with Arg191 and His192.

According to some of any of the embodiments described herein, the compound further comprises at least one additional functional moiety that is capable of interfering with an interaction between at least one amino acid residue of the calmodulin N-lobe that interacts with the linker S4-S5 of the SK4 channel, the compound being such that when it interacts with the SK4 channel, it is spatially arranged such that the at least two functional moieties that are capable of forming hydrogen bonds and/or π-π stacking interactions are in proximity and orientation that enable formation of hydrogen bonds and/or π-π stacking interactions with Arg191 and His192 and the additional functional moiety is in proximity and orientation that enable steric hindrance of Met76 of calmodulin and/or formation of hydrogen bonds and/or hydrophobic interaction with Met72 of calmodulin.

According to some of any of the embodiments described herein, the compound is capable of allosterically interfering with an interaction of Arg352 of the SK4 channel and calmodulin.

According to some of any of the embodiments described herein, the compound is represented by Formula I:

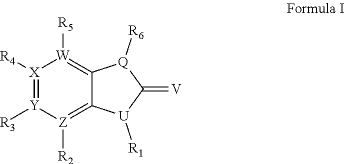

or a pharmaceutically acceptable salt thereof,
wherein:
X, Y, Z and W are each independently carbon or nitrogen, wherein when Z is nitrogen R₂ is absent; when Y is nitrogen, R₃ is absent; when X is nitrogen, R₄ is absent and when W is nitrogen, R₅ is absent;
Q, and U are each independently selected from O, S and N, wherein when Q is O or S, R₆ is absent; and when U is O or S, R₁ is absent; at least one of Q and U being nitrogen (N);
V is O, S or NR₇;
R₁, R₆ and R₇, when present, are each independently selected from hydrogen, alkyl, alkenyl, alkynyl, cycloalkyl, heteroalicyclic, aryl, heteroaryl, hydroxy, alkoxy, thiol, carboxylate, thiocarboxylate, thiohydroxy, carbonyl, carbamate, provided that one of R₁ and R₆ is an alkyl of at least 5 carbon atoms in length; and that when Q and U are each nitrogen, only one of R₁ and R₆ is the alkyl of at least 5 carbon atoms in length; and
R₂, R₃, R₄ and R₅ are each independently selected from hydrogen, alkyl, alkenyl, alkynyl, aryl, heteroaryl, halo, hydroxy, thiol, alkoxy, aryloxy, amine, amide, azo, azide, cyano, carbamyl, hydrazine, ether, ester, carbonyl, sulfonyl, sulfinyl, sulfonamide, thionyl, thioalkoxy, thioalkyl, thioaryl, thioester, thiocarbamide, thiocarbamate.

According to some of any of the embodiments described herein, V is O.

According to some of any of the embodiments described herein, U is N and R₁ is the alkyl of at least 5 carbon atoms in length.

According to some of any of the embodiments described herein, Q is N, and R₆ is hydrogen or an alkyl of up to 3 carbon atoms in length.

According to some of any of the embodiments described herein, Q is O.

According to some of any of the embodiments described herein, R₂, R₃, R₄ and R₅ are each hydrogen.

According to some of any of the embodiments described herein, R₂, R₃, R₄ and R₅ are each independently selected from hydrogen, alkyl, halo, nitro, cyano, alkoxy and thioalkoxy.

According to some of any of the embodiments described herein, X, Y, W and Z are each carbon.

According to some of any of the embodiments described herein, X, Y, W and Z are each carbon; R₂, R₃, R₄ and R₅ are each hydrogen; and V is O.

According to some of any of the embodiments described herein, U is N; R₁ is the alkyl of at least 5 carbon atoms in length; Q is N; and R₆ is hydrogen or an alkyl of up to 3 carbon atoms in length.

According to some of any of the embodiments described herein, U is N; R₁ is the alkyl of at least 5 carbon atoms in length; and Q is O.

According to an aspect of some embodiments of the present invention there is provided a compound as described herein in any of the respective embodiments and any combination thereof, for use in treating a medical condition associated with overexpression and/or overactivity of SK4 channel.

According to some of any of the embodiments described herein, the medical condition is associated with cardiac arrhythmia.

According to some of any of the embodiments described herein, the medical condition is an atrial arrhythmia.

According to some of any of the embodiments described herein, the medical condition is a ventricular arrhythmia.

According to some of any of the embodiments described herein, the medical condition is CPVT.

According to some of any of the embodiments described herein, the medical condition is myocardial infarction (MI).

According to some of any of the embodiments described herein, the medical condition is fibrosis, for example, cardiac fibrosis.

According to some of any of the embodiments described herein, the subject is a human subject.

According to some of any of the embodiments described herein, the compound forms a part of a pharmaceutical composition which further comprises a carrier.

According to an aspect of some embodiments of the present invention there is provided a compound represented by Formula I:

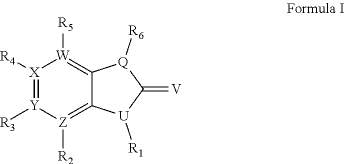

or a pharmaceutically acceptable salt thereof, as described herein in any of the respective embodiments and any combination thereof.

According to an aspect of some embodiments of the present invention there is provided a pharmaceutical composition comprising the compound as described herein in any of the respective embodiments and any combination (e.g., a compound represented by Formula I), and a pharmaceutically acceptable carrier.

According to an aspect of some embodiments of the present invention there is provided a method of identifying a candidate compound that is capable of downregulating an activity of SK4 channel, the method comprising: computationally docking a library of compounds into a calmodulin-PIP2 binding domain of an SK4 channel; and determining if a compound is arranged such that it interacts with one or more amino acid residues in the binding domain, wherein a compound that is arranged such that it interacts with the one or more amino acid residues in the binding domain is identified as a candidate compound for downregulating an activity of SK4 channel.

According to some of any of the embodiments described herein, at least one of the amino acid residues is selected from Arg191 and His192 of the SK4 channel.

According to some of any of the embodiments described herein, at least one of the amino acid residues is selected from Arg191 and His192 of the SK4 channel, and Met76 and Met72 of calmodulin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E present data obtained in inside-out macro-patch recordings, demonstrating the role of both calcium and PIP2 in SK4 K⁺ channel gating.

FIG. 1A presents comparative plots showing representative traces of wild-type (WT) SK4 currents recorded from a transfected CHO cell exposed to different intracellular free-Ca²⁺ concentrations under inside-out patch-clamp configuration. Currents are recorded by 10 repetitive 1 second duration voltage ramps from −100 millivolt (mV) to +100 mV from a holding potential of 0 mV.

FIG. 1B presents comparative plots showing representative traces of WT SK4 currents before (black) and after (red) poly-L-lysine (PLL) 50 μg/ml internal application.

FIG. 1C is a bar graph showing internal application of PLL (50 μg/ml) decreases the SK4 current by 68% (n=22; two-tailed paired t-test, t=15.71, df=21, P<0.0001).

FIG. 1D presents comparative plots showing that WT SK4 current is enhanced in response to increasing diC8-PIP2 concentrations after prior depletion of endogenous PIP2 by PLL. The experiment is performed under internal 1 μM free-Ca²⁺ concentration.

FIG. 1E presents comparative plots showing the dose-dependent activation of WT SK4 channels by internal application of diC8-PIP2, yielding an EC₅₀ of 154 nM (n=6);

FIGS. 2A-F present the PIP2-calmodulin interface and SK4 channel activation.

FIG. 2A is a bar graph showing the effect of increased PIP2 levels by co-transfection with PIP4,5-kinase on WT and mutant SK4 channels. Whole-cell SK4 K⁺ currents were activated using a voltage ramp protocol from −100 mV to +60 mV for 150 milliseconds (ms). bPIP4,5-kinase significantly increases WT SK4 and mutant Q353A currents by 3.7- and 3.5-fold, respectively (n=41 and n=29, respectively; one-way ANOVA F=4.981, Sidak's multiple comparisons test P<0.0001); all other mutants were not activated by PIP4,5-kinase (n=5-36).

FIG. 2B is a bar graph showing SK4 current density in the presence of WT CaM and purified CaM T79D (3 μM) (n=10-11, two-tailed unpaired t-test, t=2.099, df=19 P<0.0454).

FIG. 2C is a bar graph showing the current density of WT SK4 and of SK4 channels co-transfected with CK2 a subunit enzyme (n=9-12, two-tailed unpaired t-test, t=2.433, df=19, P=0.0250).

FIG. 2D is a bar graph showing the effect of PIP4,5-kinase on WT CaM, and on the CaM mutants K75A and T79D (n=8-19).

FIG. 2E presents simulation of PIP2 (green stick) docking to the Ca²⁺-bound state I (6CNN) of the SK4 channel cryo-EM structure; the S1-S4 helices, the CaM and the SK4 proximal C-terminus (helices A and B) are shown in deep purple, grey, and cyan, respectively.

FIG. 2F presents simulation of the interactions between PIP2 and specific amino acid residues in the PIP2 binding pocket of SK4.

FIGS. 3A-C present the effects of exemplary tested compounds on SK4 channel activation.

FIG. 3A presents comparative plots showing representative trace of WT SK4 currents in the absence and presence of 10 μM BA40, an exemplary tested compound, indicating activation of SK4 channel by about 1.4-fold. Whole-cell SK4 K⁺ currents are activated using a voltage ramp protocol from −100 mV to +60 mV for 150 milliseconds.

FIG. 3B presents comparative plots showing representative trace of WT SK4 currents in the absence and presence of 20 μM BA6b, an exemplary tested compound, indicating inhibition of SK4 channel by about 25%. Whole-cell SK4 K⁺ currents are activated using a voltage ramp protocol from −100 mV to +60 mV for 150 milliseconds.

FIG. 3C is a bar graph presenting statistical summary of the pharmacological effects of 10 μM of an exemplary tested compound, BA40 or BA100, and 20 μM of BA6b (an exemplary compound according to some of the present embodiments) on WT SK4 currents, showing 42% activation (n=14), 28% activation (n=9), and 25% inhibition (n=6), respectively.

FIGS. 4A-C present the inhibitory influence of the exemplary compound BA6b9 on SK4 K⁺ channel.

FIG. 4A presents comparative plots showing the dose-dependent activation of WT SK4 channels by intracellular free-Ca²⁺ in the presence (n=5) and absence (n=6) of 10 μM BA69b, an exemplary compound according to some of the present embodiments, yielding EC₅₀ of 435 nM and 65 nM, respectively.

FIG. 4B shows a declining plot showing the dose-dependent inhibition of WT SK4 channels by BA69b, an exemplary compound according to some of the present embodiments, yielding an IC₅₀ of 8.6 μM (n=6).

FIG. 4C presents comparative plots showing representative trace of WT SK4 currents in the absence and presence of 20 μM BA6b9, an exemplary compound according to some of the present embodiments, indicating inhibition of SK4 channel by about 56%. Whole-cell SK4 K⁺ currents are activated using a voltage ramp protocol from −100 mV to +60 mV for 150 milliseconds.

FIGS. 5A-C present the molecular docking of PIP2 and tested compounds to the PIP2-binding domain of SK4 K⁺ channel.

FIG. 5A presents simulation of the PIP2 docking pose in the PIP2-binding pocket of SK4 channel, indicating that BA6b9 (orange stick), an exemplary compound according to some of the present embodiments, fits into a gorge formed by the boundaries of S1 and S4 helices and the S4-S5 linker (deep purple) in close proximity to the bound PIP2 (green stick); the CaM and the SK4 proximal C-terminus (helices A and B) are shown in grey and cyan, respectively.

FIG. 5B presents simulation of the specific interactions between BA6b9, an exemplary compound according to some of the present embodiments, and the residues near the PIP2 binding pocket of SK4. BA6b9 is docked to the Ca²⁺-bound state I (6CNN) of the SK4 channel cryo-EM structure; BA6b9, PIP2, S1-S4 helices, CaM and the SK4 proximal C-terminus (helices A and B) are represented;

FIG. 5C presents simulation of the molecular docking of BA6b9 (an exemplary compound according to some of the present embodiments), 1-EBIO and BA40 to the SK4 channel. Docking was performed to the Ca²⁺-bound state I (6CNN) of the SK4 channel cryo-EM structure; the S1-S4 helices and the SK4 proximal C-terminus (helices A and B) are shown in deep purple and cyan, respectively. PIP2 is shown in deep teal stick and BA6b9, 1-EBIO, and BA40 are displayed in green sticks;

FIGS. 6A-B present functional validation of the interaction of BA6b9, an exemplary compound an exemplary compound according to some of the present embodiments, with residues R191 and H192.

FIG. 6A is a bar graph showing inhibition by BA6b9, an exemplary compound according to some of the present embodiments, on the activity of WT SK4 channel compared to several SK4 channel mutants, indicating the mutants R352Q, R191A, and H192A are significantly less sensitive to the inhibitory effect of 20 μM BA6b9 compared to WT SK4 (one-way ANOVA, F(10, 165)=10.27; Dunnett's multiple comparisons test; n=14, P<0.0003, n=16, p<0.0001 and n=11, p<0.0001, and n=62, respectively).

FIG. 6B presents comparative plots showing representative trace of the SK4 mutant H192A current in the absence and presence of 20 μM BA6b9, an exemplary compound according to some of the present embodiments, indicating decreased inhibition (16%) compared to that obtained for WT SK4 (56%);

FIGS. 7A-C present the effect of BA6b9, an exemplary compound according to some of the present embodiments, on the SK channel family members SK1-SK3.

FIG. 7A shows multiple protein sequence alignment of human SK1-SK3 channels compared to SK4 (T-coffee server: http://tcoffee(dot)crg(dot)cat/apps/tcoffee/do: tmcoffee) indicating that the amino acid sequence is not conserved at the S4-S5 linker region in SK4 channel, such that the residues R191 and H192 of the SK4 channel are different from the respective residues of the SK1-SK3 channel isoforms.

FIG. 7B presents comparative plots showing representative trace of human SK1 currents in the absence and presence of 20 μM BA6b9, an exemplary compound according to some of the present embodiments, showing that the tested compound has nearly nullified effect in this channel;

FIG. 7C presents comparative bar plots, showing statistical summary of the pharmacological effects of 20 μM BA6b9, an exemplary compound according to some of the present embodiments, on the SK channel family members SK1-SK3, indicating it does not affect human SK1, rat SK2, and human SK3 with 118%, 134%, and 95% activity in comparison with control, respectively.

FIGS. 8A-B present functional validation of the molecular docking of BA6b9, an exemplary compound according to some of the present embodiments, to CaM amino acid residues M72 and M76.

FIG. 8A is a bar graph showing statistical summary of the pharmacological effects of 20 μM BA6b9, an exemplary compound according to some of the present embodiments, on WT SK4 channel co-transfected with WT CaM and with CaM mutants M72A and M76A, demonstrating the lower sensitivity of both mutants towards inhibition by BA6b9 (one-way ANOVA F=25.59, Dunnett's multiple comparisons test; respectively, 0% inhibition n=7, P<0.0001 and 19% inhibition n=7, P=0.0008) as compared to WT CaM (47% inhibition, n=24).

FIG. 8B presents comparative plots showing representative trace of WT SK4 channel co-transfected with CaM mutant M72A in the absence and presence of 20 μM BA6b9, an exemplary compound according to some of the present embodiments, not indicating modulation by this compound.

FIG. 8C presents comparative plots showing representative traces of an inside-out macro-patch from a CHO cell expressing WT SK4 channels in the absence and presence of 10 μM BA6b9, an exemplary compound according to some of the present embodiments, under internal 1 μM free-Ca²⁺ concentration. Currents are recorded by 10 repetitive 1 second duration voltage ramps from −100 mV to +100 mV from a holding potential of 0 mV.

FIGS. 9A-E present the effects of BA6b9, an exemplary compound according to some of the present embodiments, on the cardiac conduction system of isolated rat hearts. Data are analyzed by two-tailed paired t-test, except in FIG. 9D, by two-tailed Mann Whitney test.

FIG. 9A is a bar graph showing that AERP and AVERP are prolonged by 10 μM BA6b9, an exemplary compound according to some of the present embodiments (n=16, t=2.822, df=15, P=0.0129 and n=15, t=4.706, df=14, P=0.0003, respectively).

FIG. 9B is a bar graph showing the effect of 10 μM BA6b9, an exemplary compound according to some of the present embodiments on heart rate (n=19, t=6.209, df=18, P<0.0001).

FIG. 9C is a bar graph showing the effect of 10 μM BA6b9, an exemplary compound according to some of the present embodiments, on PR interval (n=17, t=5.592, df=16, P<0.0001).

FIG. 9D is a bar graph showing the effect of 10 μM BA6b9, an exemplary compound according to some of the present embodiments, on AFIS (AF induction score, see Materials and Experimental Methods), compared to carbachol alone (n=7, P=0.0023; and n=10, P=0.0218, respectively).

FIG. 9E is a bar graph showing data from sustained AF induced by burst pacing (see: Materials and Experimental Methods) in the presence of 10 μM BA6b9, an exemplary compound according to some of the present embodiments, showing it prevents sustainability in 30% (3/10) of heart preparations.

FIGS. 10A-H show the effects of Tram-34 and BA6b9, an exemplary compound according to some of the present embodiments, on the cardiac conduction system of isolated guinea pig hearts. Data are analyzed by two-tailed paired t-test.

FIGS. 10A and 10B are bar graphs presenting data obtained in refractory period measurements as described herein, in the presence of Tram-34 (FIG. 10A) and BA6b9, an exemplary compound according to some of the present embodiments (FIG. 10B). AERP and AVERP are significantly prolonged in the presence of 10 μM Tram-34 (n=10, t=2.462, df=9, P=0.036 and t=2.892, df=9, P=0.0178, respectively) and 10 μM BA6b9 (n=9, t=5.382, df=8, P=0.0007 and t=2.680, df=7, P=0.0315, respectively); in the two rightmost bar plots VERP is significantly prolonged in the presence of 10 μM BA6b9 (n=9, t=4.789, df=8, P=0.0014).

FIGS. 10C and 10D are bar graphs showing the effect of 10 μM Tram-34 (n=10, t=2.947, df=9, P=0.0163; FIG. 10C) and of 10 μM BA6b9, an exemplary compound according to some of the present embodiments (n=9, t=5.774, df=8, P=0.0004; FIG. 10D), on heart rate.

FIGS. 10E and 10F are bar graphs showing the effect of 10 μM Tram-34 (FIG. 10E) and of 10 μM BA6b9 (n=9, t=5.778, df=8, P=0.0004; FIG. 10F) on PR interval.

FIGS. 10G and 10H are bar graphs showing the effect of 10 μM Tram-34 (FIG. 10G) and 10 μM BA6b9, an exemplary compound according to some of the present embodiments (FIG. 10H) on the atrial and ventricular pacing thresholds.

FIGS. 11A-H show the effects of Tram-34 and BA6b9, an exemplary compound according to some of the present embodiments, on hemodynamic parameters in isolated rat hearts. Data are analyzed by two-tailed paired t-test.

FIGS. 11A and 11B are bar graphs showing the effect of 10 μM Tram-34 (FIG. 11A) and of 10 μM BA6b9, an exemplary compound according to some of the present embodiments (FIG. 11B) on the left ventricular filling pressure (n=6, t=2.626, df=5, P=0.0468).

FIGS. 11C and 11D are bar graphs showing the effect of 10 μM Tram-34 (FIG. 11C) and of 10 μM BA6b9, an exemplary compound according to some of the present embodiments (FIG. 11D) on the maximal pressure change in left ventricular contractility (dP/dt max; n=10, t=2.613, df=9, P=0.0281).

FIGS. 11E and 11F are bar graphs showing the effect of 10 μM Tram-34 (FIG. 11E) and of 10 μM BA6b9, an exemplary compound according to some of the present embodiments (FIG. 11F) on the minimal pressure change in left ventricular contractility (dP/dt min; n=10, t=3.549, df=9, P=0.0062).

FIGS. 11G and 11H are bar graphs showing the effect of 10 μM Tram-34 (FIG. 11G) and of 10 μM BA6b9, an exemplary compound according to some of the present embodiments (FIG. 11H) on the perfusion flow velocity, a marker for coronary perfusion pressure (n=12, t=5.126, df=11, P=0.0003).

FIGS. 12A and 12B are bar graphs showing the effects of Tram-34 (FIG. 12A) and BA6b9, an exemplary compound according to some of the present embodiments (FIG. 12B), on perfusion flow velocity in isolated guinea pig hearts.

FIGS. 13A-D are bar graphs presenting data obtained in in-vivo rat model of heart failure (HF) post-myocardial infarction (MI) as described herein, one week following MI (denoted as “base”) and following a subsequent 2-week treatment (denoted as “final”) with an exemplary compound according to some of the present embodiments, BA6b9 (20 mg/kg/day; grey bars), or with vehicle (white bars; control). FIG. 13A presents EF data; FIG. 13B presents AERF data; FIG. 13C presents AF induction; and FIG. 13D presents total AF duration.

FIGS. 14A-B are images of Masson-trichrome staining of left atrial (LA) sections in rats following treatment with vehicle (FIG. 14A) or with BA6b9 (FIG. 14B), as described in FIGS. 13A-D.

FIGS. 14C-D are bar graphs presenting quantitative analyses of LA fibrosis (FIG. 14C) and LA smooth muscle actin (SMA) (FIG. 14D) in rats following treatment with vehicle (orange) or with the exemplary compound BA6b9 (green), as described in FIGS. 13A-D.

### DESCRIPTION OF SPECIFIC EMBODIMENTS OF THE INVENTION

The present invention, in some embodiments thereof, relates to therapy and, more particularly, but not exclusively, to novel compounds that modulate (e.g., downregulate) an activity of Ca²⁺-activated potassium channel SK4, which are usable in the treatment of arrhythmic disorders and other medical conditions that are associated with SK4 activity and/or in which downregulating an activity of SK4 is beneficial.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As delineated in the Background section hereinabove, Ca²⁺-activated potassium SK4 channels are expressed in a variety of cells. Although exhibiting similar design as the small-conductance potassium channels, SK4 K⁺ channels are gated by Ca²⁺-bound calmodulin (CaM), which is tethered to a CaM-binding domain (CaMBD) located at the proximal C-terminus of the SK4 channel, and contacting the S4-S5 intracellular linker.

In-vivo studies have shown that blockage of SK4 channels affects cardiac function, e.g., reduces the risk of atrial fibrillation (AF) in canine hearts. While AF is the most common sustained cardiac arrhythmia and affects more than 4% of the global population, currently available treatment methods for AF have major limitations.

Known SK4 channel inhibitors, such as clotrimazole and Tram-34, focus mainly on the channel pore. However, they were deemed inappropriate for clinical trials due to high liver toxicity and poor bioavailability. The present inventors therefore sought to identify new SK4 channel modulators, and specifically SK4 channel inhibitors (or blockers), for the treatment of various conditions associated with SK4 channel activity, such as, but not limited to, atrial fibrillation.

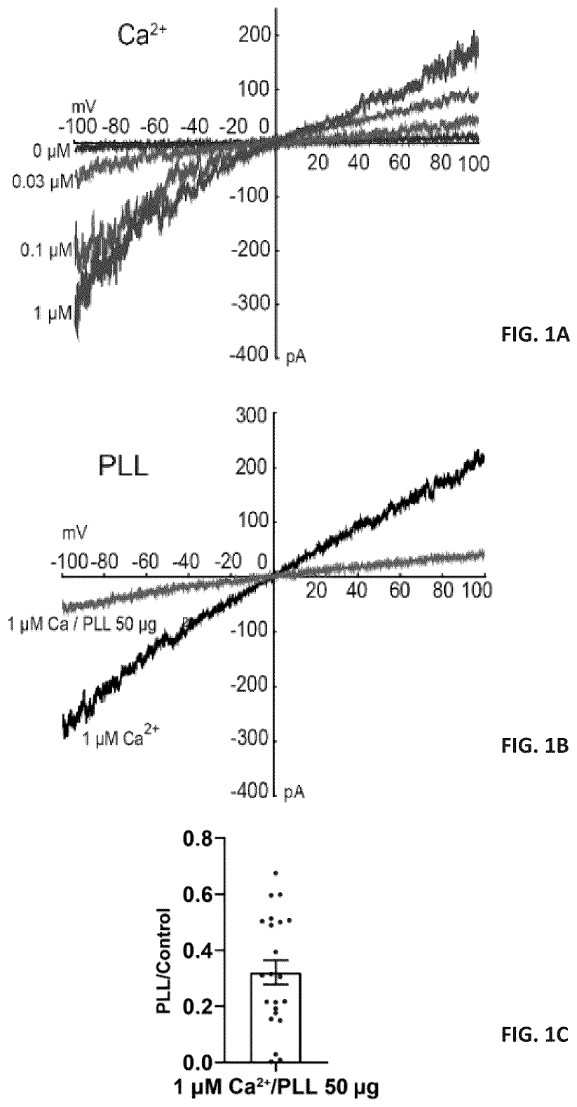

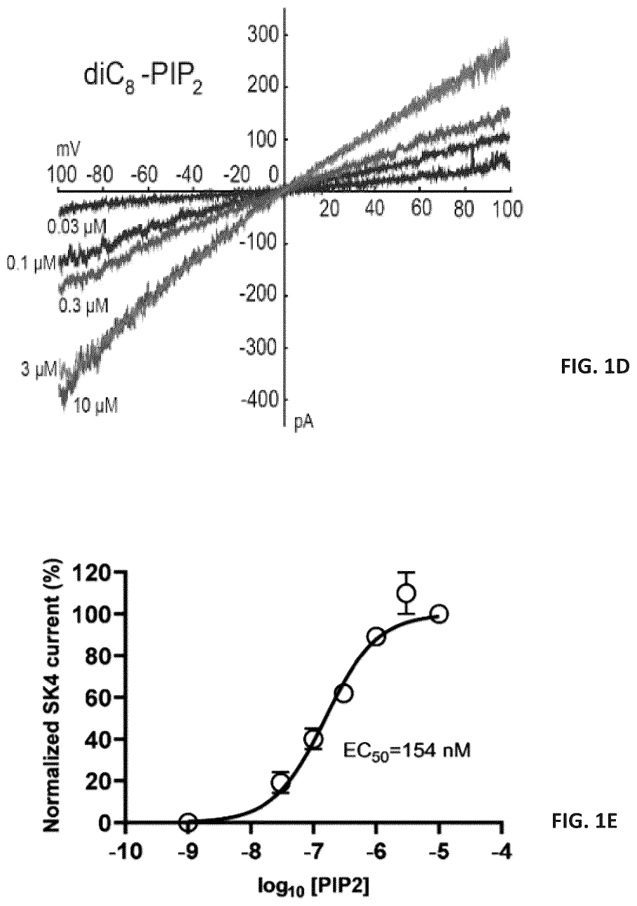

As described in the Examples section that follows, the present inventors have uncovered that PIP2 is an activator of SK4 K⁺ channels, and have identified its molecular binding pocket in an allosteric site of the SK4 channel (see, e.g., FIGS. 1A-E and 2A-F and Example 1).

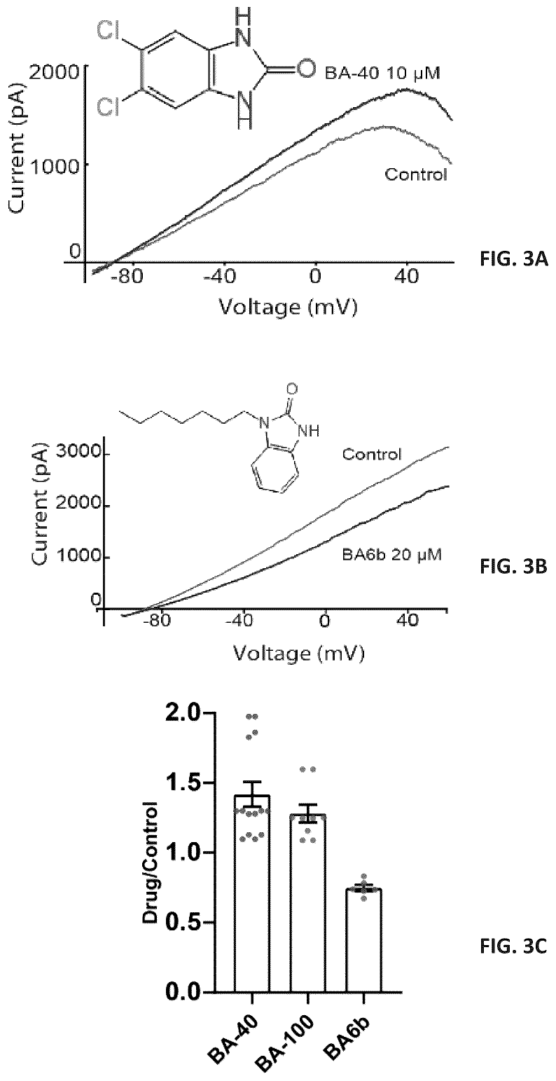

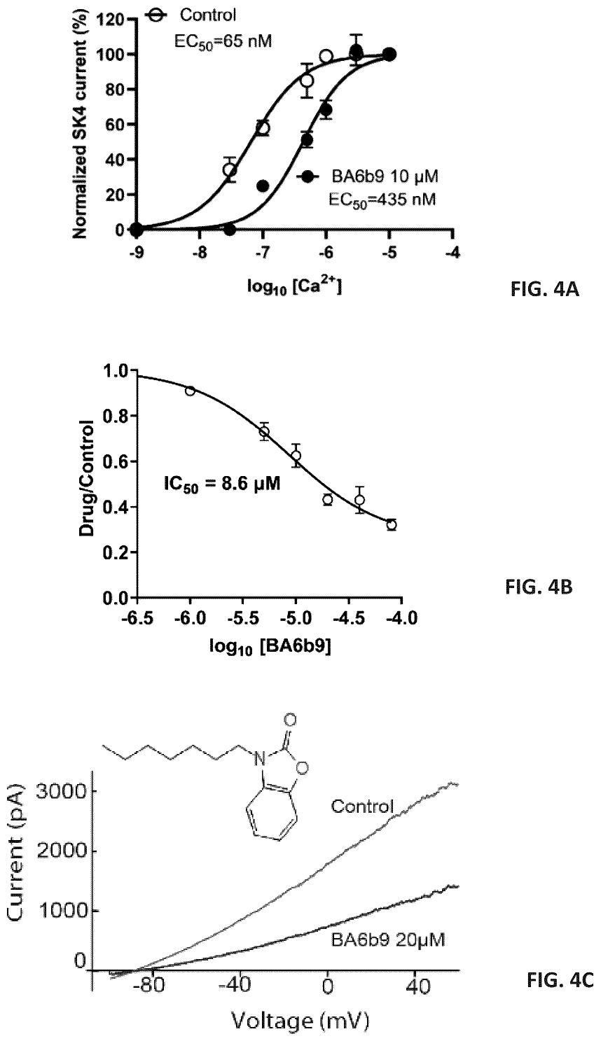

Based on these findings, the present inventors have followed a trans-disciplinary approach to discover novel small molecules that can interfere with (i.e., interrupt, disrupt or block) the interaction between PIP2 and the SK4 channel. By manipulating structures of known compounds that activate SK channels by binding in proximity to the PIP2-binding domain of the SK4 channel, benzimidazole- and benzoxazole-based small molecules were designed and synthesized (see, e.g., Tables 1 and 2). The effect of the small molecules on the activity of the SK4 channel was studied (Table 3), and data indicated that halide-bearing exemplary small molecules (e.g., BA40, BA100) activate SK4 channels, while mono-N-alkylated exemplary small molecules (e.g., BA6B, BA6b9) inhibit SK4 channel activity (see, e.g., FIGS. 3A-4C).

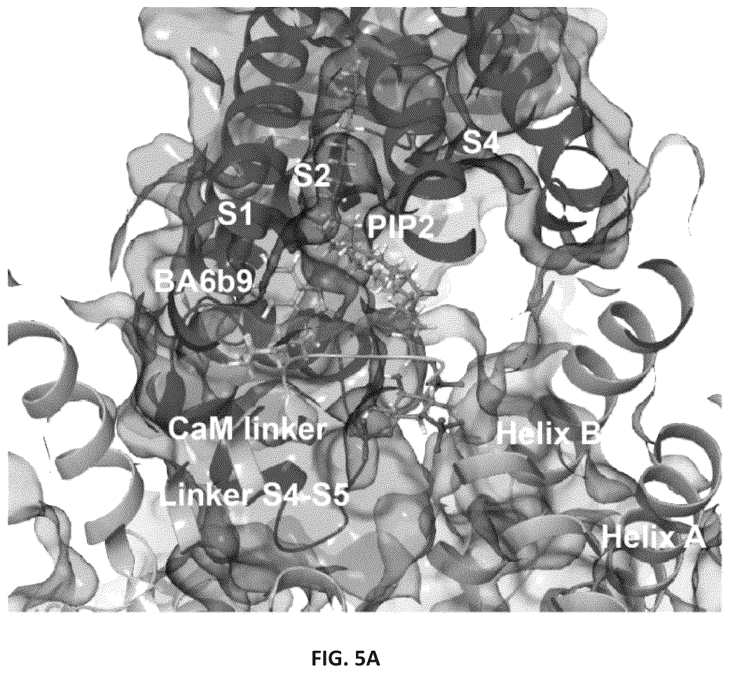

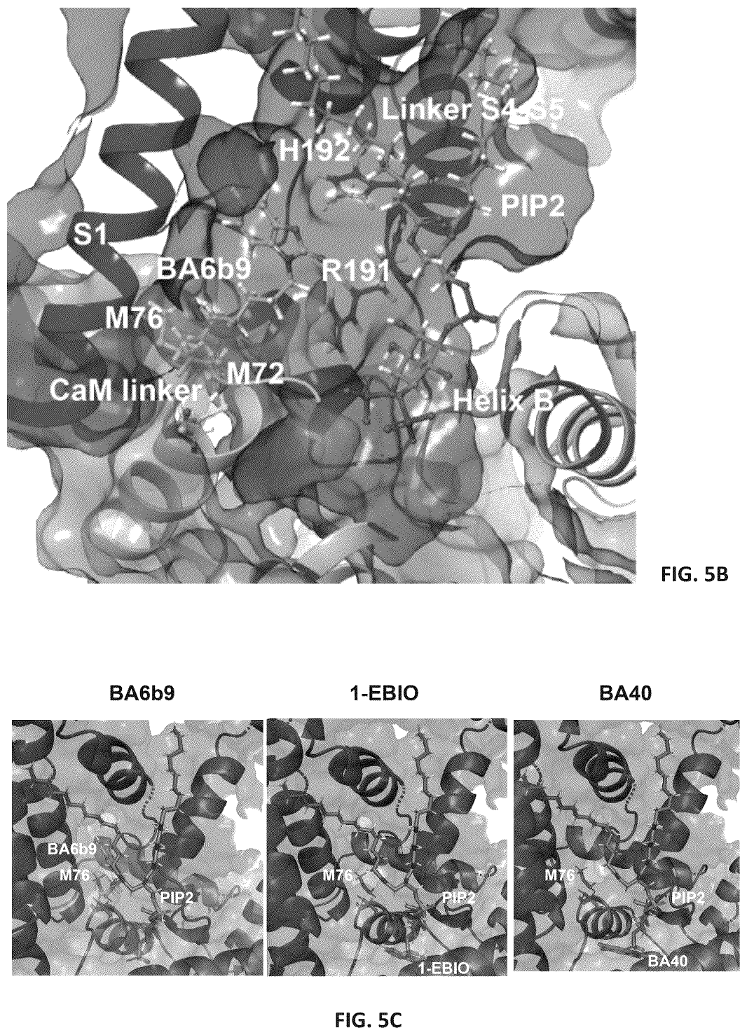

The docking of an exemplary compound BA6b9 was simulated in the presence of PIP2 (see, FIGS. 5A-C), and suggested it forms H-bonding and π-π stacking interactions with the calmodulin-PIP2 binding domain (CPBD) of the SK4 channel, a region located at a boundary of the channel proximal C-terminus and the S4-S5 linker.

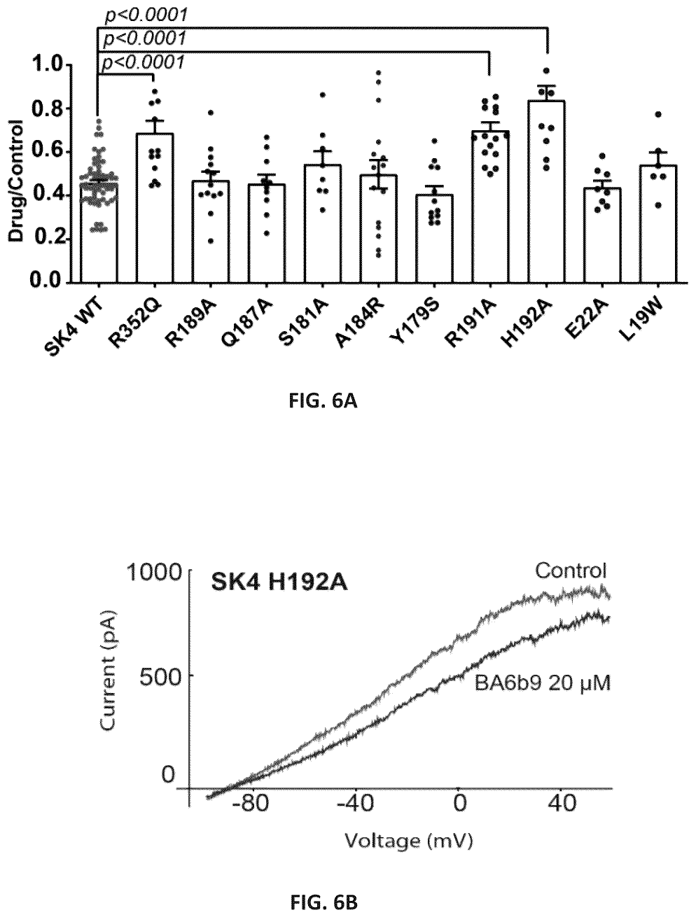

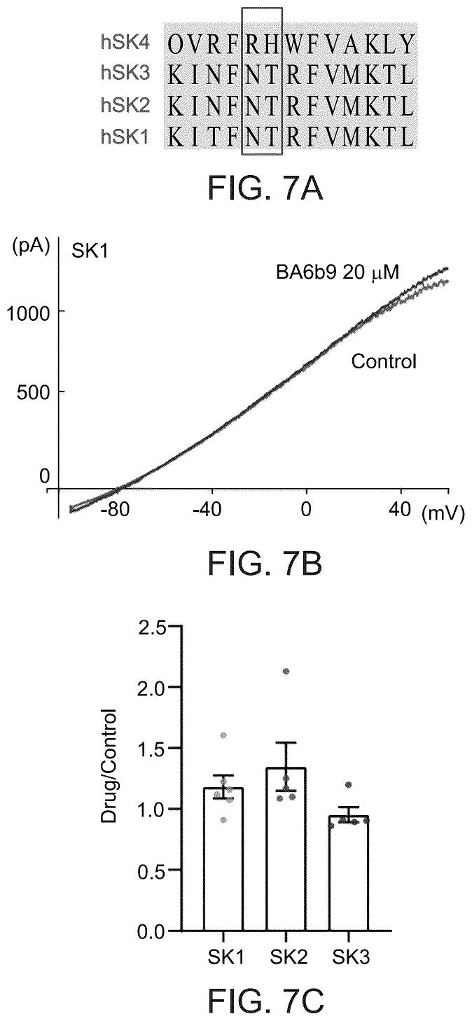

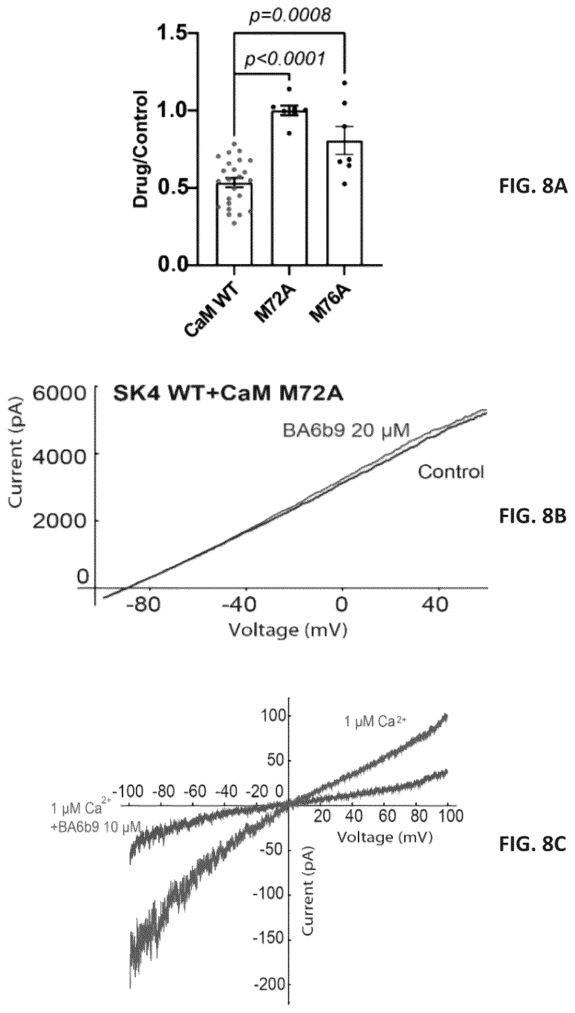

Ex-vivo studies have revealed that the newly designed compounds allosterically inhibit SK4 channel gate opening by interacting with residues R191 and H192 of the SK4 channel (see, e.g., FIGS. 6A-B) and with residues M72 and M76 of CaM (see, e.g., FIGS. 8A-B). As residues R191 and H192 are not conserved in SK1-SK3 channels subunits, selectivity towards SK4 channel was demonstrated and indicated the role of these residues in the inhibition mechanism of the SK4 channel by the newly designed compounds (see, e.g., FIGS. 7A-C). Inhibition of SK4 currents by these compounds (at an exemplary concentration of 10 μM) has been demonstrated (e.g., FIG. 8C).

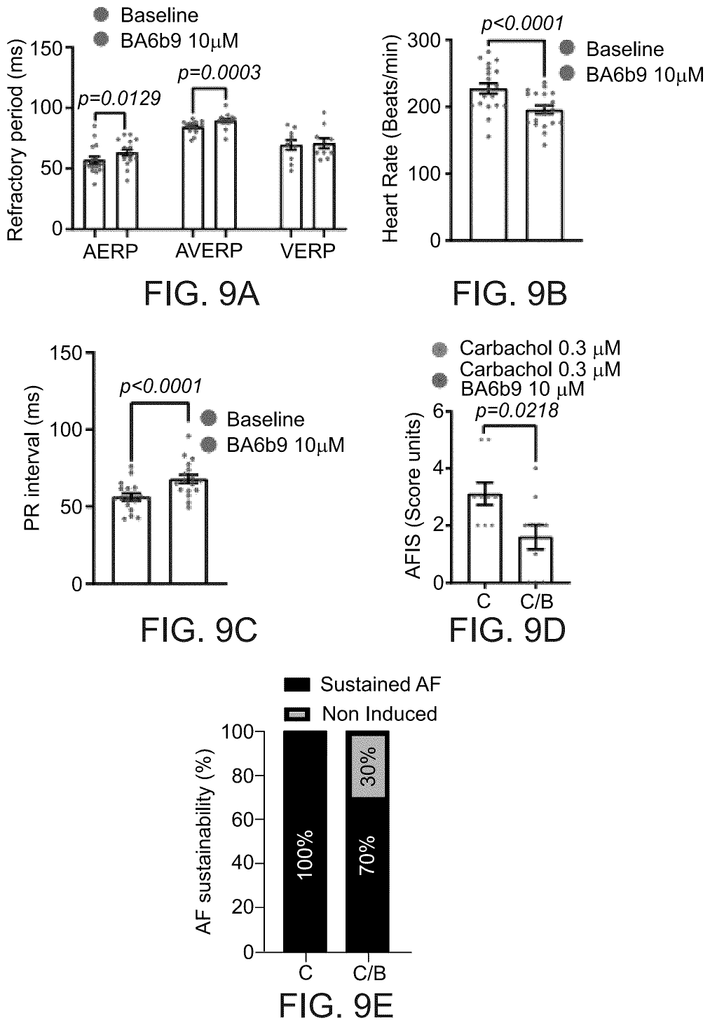

The newly designed compounds were further shown to prolong AERP and AVERP, decrease heart rate and increase PR interval ex-vivo (see, e.g., FIGS. 9A-12B and Example 4).

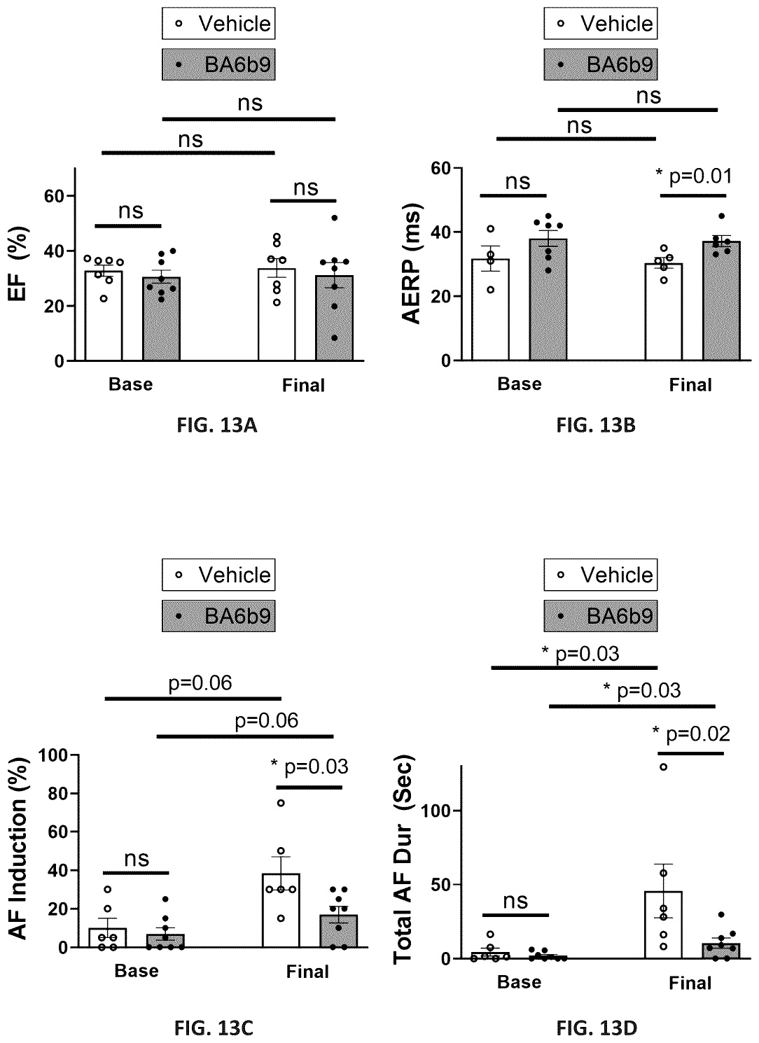

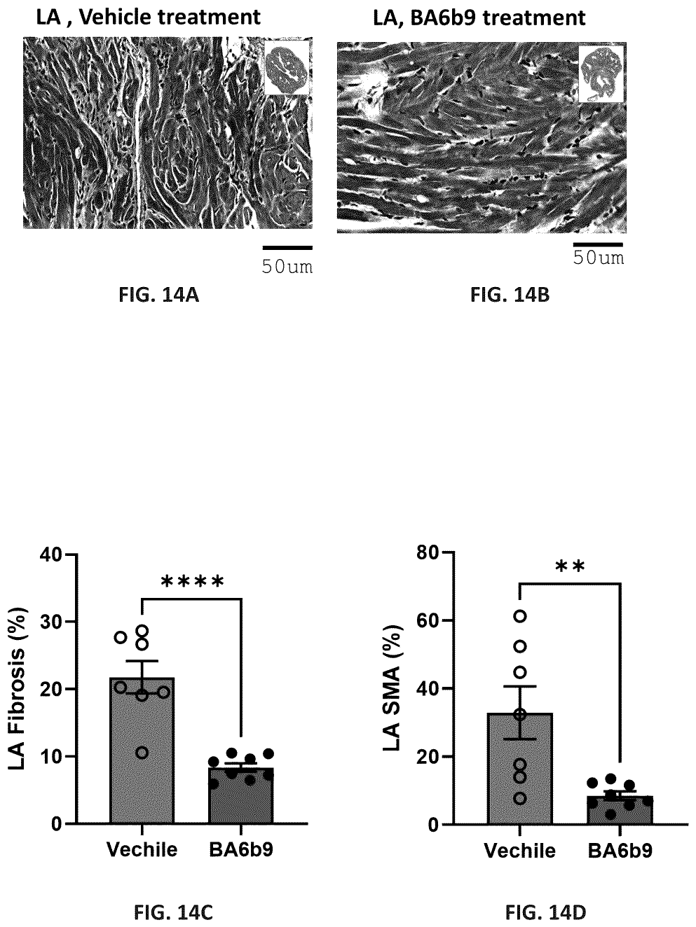

The newly designed compounds were further tested in-vivo on myocardial infarction (MI) in rats, and successfully reduced AF substrate in post-MI rats (see, e.g., FIGS. 13A-D and Example 5) and ameliorated structural remodeling (see, e.g., FIGS. 14A-D), thus demonstrating a promising use of these compounds in the treatment of both MI and cardiac fibrosis.

Embodiments of the present invention therefore relate to newly designed compounds which downregulate SK4 activity by targeting the CPBD of an SK4 channels. Embodiments of the present invention also relate to methods of screening and identifying lead candidate compounds that are capable of downregulating an activity of SK4 channel, as described herein, by determining interference or blockade of a calmodulin-PIP2 binding domain of an SK4 channel by the screened library of compounds.

Herein throughout, the phrases “SK4 channel” “SK4 K⁺ channel”, “SK4 potassium channel”, “Ca²⁺-activated potassium channel SK4”, and similar phrases that relate to SK4 channel are used interchangeably and describe the intermediate-conductance calcium-activated potassium channel K_{Ca}3.1, which is also referred to in the art as IK1 channel or SK4 channel. In some embodiments, the SK4 channel is a human SK4 channel, or is analogous to a human SK4 channel, that is, exhibits at least 50%, at least 60%, at least 70%, or at least 80%, homology to a human SK4 channel.

According to an aspect of some embodiments of the present invention there is provided a compound for use in downregulating an activity of an SK4 channel in a subject in need thereof. According to embodiments of the present invention the compound is such that is capable of interacting with the calmodulin-PIP2 binding domain of the SK4 channel to thereby interfere with the Ca²⁺-dependent activation of the SK4 channel.

According to an aspect of some embodiments of the present invention there is provided a method of downregulating an activity of an SK4 channel in a subject in need thereof, the method comprising administering to the subject a compound that is capable of interacting with the calmodulin-PIP2 binding domain of the SK4 channel to thereby interfere with the Ca²⁺-dependent activation of the SK4 channel, as described herein in any of the respective embodiments and any combination thereof.

Herein, the term “downregulating an activity” and grammatical diversions thereof describe reducing, inhibiting, inactivating or blocking the SK4 channel, for example, by reducing or inhibiting SK4 channel function as a channel of potassium ions (i.e., a channel that allows potassium ions to cross the cell membrane).

Reduction or Inhibition of SK4 channel function, as used herein, can be manifested as reducing or inhibiting the function of the channel by at least 10%, preferably by at least 20%, or at least 30%, or at least 40%, or at least 50%, or at least 60%, or at least 70%, or at least 80%, or at least 90% and in some embodiments, by 95%, 96%, 97%, 98%, 99% or even 100%.

Reduction or inhibition of SK4 channel function is manifested, for example, by a reduction in the electrical current produced by the channel as is further described in the Example section that follows (as illustrated, e.g., in FIG. 8C), and can be determined using methods known in the art, e.g., as described herein.

In some embodiments, determining if a SK4 current amplitude is reduced or inhibited is affected by measuring the SK4 current amplitude, or measuring a change in the SK4 current amplitude, upon contacting a tested compound compared with the SK4 current amplitude in the absence of the tested compound.

Determining if a compound downregulates the activity of SK4 channel can be performed using methods known in the art, some are described hereinafter in the context of the screening method. Other methods are readily recognized by those skilled in the art.

In some embodiments, a compound that downregulates the activity of SK4 is capable of blocking other calcium ion-activated channel and/or or a potassium channel. In preferred embodiments, a compound that downregulates the activity of SK4 channel is selective towards SK4 channel.

Currently known SK4 inhibitors include, for example, Clotrimazole (1-[(2-chlorophenyl)diphenylmethyl]-1H-imidazole) and Tram-34 (1-[(2-chlorophenyl)diphenylmethyl]-1H-pyrazole):

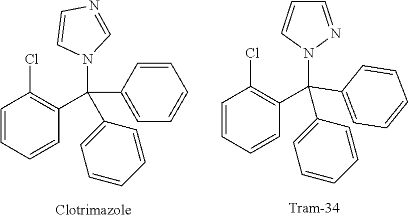

Herein throughout, the term “calmodulin” is used interchangeably with “CaM”, and describes a multifunctional intermediate messenger protein, which is activated upon binding of calcium ions, as known in the art. Calmodulin participates as a subunit of the channel, and is bound (i.e., tethered) via a linker (i.e., the CaM linker) to the cytoplasmic C-terminus region of the SK4 channel called the calmodulin binding domain (CaMBD or CMBD). The calcium-activated form of calmodulin is also referred to herein as “Ca²⁺-bound calmodulin” or “calcified calmodulin”.

The term “PIP2” or “PI(4,5)P2” (1,2-Diacyl-sn-glycero-3-phospho-(1-D-myo-inositol 4,5-bisphosphate)) describes a phospholipid component which is known to be involved in, e.g., CaM-SK complex activation in SK1-SK3 potassium channels.

As used herein, the term “calmodulin-PIP2 binding domain” and grammatically diversions thereof describe a domain which is in close proximity to the CaM linker and in proximity to the domain to which PIP2 binds in the SK4 channel.

Herein, the phrases “capable of interacting with” and “interacting with” describe a compound that binds to or forms one or more molecular interactions with another molecule or molecules, e.g., an amino acid residue of the SK4 channel. Exemplary molecular interactions include Van der Waals, hydrophobic interactions, hydrogen bonds, aromatic interactions (e.g., π-π stacking), electrostatic interactions, and any a combination thereof.

In some of any of the embodiments described herein, “a compound that is capable of interacting with the calmodulin-PIP2 binding domain of the SK4 channel” is a compound that is capable of interacting, as described herein, with one or more amino acid residues that form the calmodulin-PIP2 binding domain of the SK4 channel. Amino acid sequences that form this domain in SK4 channels are known in the art.

In some of any of the embodiments described herein, a compound that is capable of interacting with the calmodulin-PIP2 binding domain of the SK4 channel as described herein, is a compound that bears a functional moiety which is capable of interacting with at least one amino acid residue of the calmodulin-PIP2 binding domain of the SK4 channel, under, e.g., physiological conditions.

In some of any of the embodiments described herein, upon interaction with the calmodulin-PIP2 binding domain of the SK4 channel, as described herein, the compound interferes with or blocks an interaction between SK4 channel and an activator thereof (e.g., PIP2). In some of any of the embodiments described herein, interfering with or blocking the SK4 channel and an activator therefore (e.g., PIP2) interferes with the Ca²⁺-dependent activation of the SK4 channel. In some of any of the embodiments described herein, interfering with the Ca²⁺-dependent activation of the SK4 channel results in the inhibition and/or inactivation of SK4 channel, as described herein.

Herein, the term “Ca²⁺-dependent activation of the SK4 channel” and grammatical diversions thereof describe the calcium-gated (i.e., calcium-mediated) channeling of potassium ions by the SK4 channel, as known in the art.

In some of any of the embodiments described herein, the calmodulin-PIP2 binding domain of the SK4 channel is a region located at a boundary of the channel proximal C-terminus and the S4-S5 linker.

According to an aspect of some embodiments of the present invention there is provided a method of downregulating an activity of an SK4 channel, the method comprising contacting cells expressing an SK4 channel, or suspected as expressing an SK4 channel, with a compound that is capable of interacting with the calmodulin-PIP2 binding domain of the SK4 channel to thereby interfere with the Ca²⁺-dependent activation of the SK4 channel, as described herein in any of the respective embodiments and in any combination thereof.

In some embodiments, the cells expressing the SK4 channels are cells inherently expressing SK4 potassium channels. In some embodiments, the cells expressing the SK4 channels are cancerous cells. In some embodiments, the cells expressing the SK4 channels are transfected cells ectopically expressing a SK4 potassium channel (e.g., by means of cDNA encoding SK4 channel).

Cells inherently expressing the SK4 channels include, but are not limited to, sinoatrial node (SAN) cells, T cells, B cells, mast cells, macrophages, and microglial cells. SK4 potassium channels are also expressed in cancerous cells, e.g., triple-negative breast cancer (TNBC).

As used herein, “suspected as expressing an SK4 channel” are cells which may or may not have been recognized in the art as cells expressing the SK4 channels.

In some embodiments, contacting with cells expressing the SK4 channel is affected in-vivo. In some embodiments, the contacting with cells expressing the SK4 potassium channel is affected in-vitro or ex-vivo.

According to some of any of the embodiments described herein, the compound is capable of interfering with an interaction of a calcified calmodulin N-lobe with a proximal S₄₅A helix of the SK4 channel.

According to some of any of the embodiments described herein, interfering with the interaction allosterically affects the Ca²⁺-dependent activation of the SK4 channel.

By “allosterically” in the context of the present embodiments it is meant that the interference occurs at a site which differs from the substrate-binding site of the SK4 channel or from the site at which activation of the SK4 channel occurs.

According to some of any of the embodiments described herein, the compound comprises at least one functional moiety that is capable of interacting with at least one amino acid residue at a boundary of the SK4 channel proximal C-terminus and the S4-S5 linker (e.g., capable of interacting with one or more amino acid residues at the location of the SK4 channel that interacts with PIP2, that is, the PIP2 binding pocket in the SK4 channel).

The term “π-π stacking” as used herein refers to a non-covalent interaction (i.e., an interaction that does not involve the sharing of electrons) involving aromatic groups containing π bonds.

In some of any of the embodiments described herein, the compound as described herein is capable of forming π-π stacking, with at least one, or at least two, amino acid residue(s) of the SK4 channel (e.g., at the PIP2 binding pocket as referred to herein). In some embodiments, the compound as described herein is aromatic or comprises at least one aromatic moiety (e.g., imidazole moiety). In some embodiments, the compound as described herein is aromatic or comprises at least one aromatic moiety and binds to an aromatic amino acid residue at the PIP2 binding pocket as referred to herein.

The term “hydrogen bonds” refers to a form of association between an electronegative atom (also known as a hydrogen bond acceptor) and a hydrogen atom attached to a second, relatively electronegative atom (also known as a hydrogen bond donor). Suitable hydrogen bond donor and acceptors are well understood in medicinal chemistry.

The term “hydrogen bond acceptor” refers to a group comprising an oxygen, nitrogen or sulfur, such as an oxygen or nitrogen that are sp²-hybridized, an ether oxygen, or the oxygen of a sulfoxide or N-oxide.

The term “hydrogen bond donor” refers to an oxygen, nitrogen, sulfur, or heteroaromatic carbon that bears, for example, a hydrogen group containing a ring nitrogen or a heteroaryl group containing a ring nitrogen.

In some of any of the embodiments described herein, the compound as described herein is capable of forming hydrogen bonds with at least one, or at least two, amino acid residue(s) of the SK4 channel (e.g., at the PIP2 binding pocket as referred to herein). In some embodiments, the compound as described herein is a hydrogen bond acceptor. In some embodiments, the compound as described herein is a hydrogen bond donor. In some embodiments, the compound as described herein is both a hydrogen bond donor and an acceptor.

In some of any of the embodiments described herein, the compound as described herein is capable of forming π-π stacking as described herein, and is capable of forming hydrogen bonding as described herein, with at least one, or at least two, amino acid residue(s) at the PIP2 binding pocket as referred to herein.

By “hydrogen bond acceptor atom or moiety” it is meant an atom or group or moiety that is capable of forming a hydrogen bond with a hydrogen atom that forms a part of an electronegative group or moiety (which acts as a hydrogen bond donor). Thus, when forming a hydrogen bond, the hydrogen atom is partially linked to the donor group or moiety and partially linked to the acceptor atom or moiety.

According to some of any of the embodiments described herein, the at least one amino acid residue is Arg191, His192 or both.

According to some of any of the embodiments described herein, the compound comprises at least two functional moieties spatially arranged such that the compound is capable of forming hydrogen bonds and/or π-π stacking (e.g., aromatic) interactions with at least two amino acid residues (e.g., Arg191 and His192) at the boundary (e.g., the PIP2 binding pocket as referred to herein) of the SK4 channel.

According to some of any of the embodiments described herein, the at least two amino acid residues comprise Arg191 and His192.

According to some of any of the embodiments described herein, the at least one or at least two functional moieties comprise at least one or at least two functional moieties that feature a hydrogen bond acceptor atom or moiety.

In some of any of the embodiments described herein, the compound as described herein is capable of forming π-π stacking as described herein, with His192 of the SK4 channel. In some of any of the embodiments described herein, the compound as described herein is capable of forming hydrogen bonding as described herein, with His192 of the SK4 channel. In some of any of the embodiments described herein, the compound as described herein is capable of forming 71-71 stacking as described herein, and is capable of forming hydrogen bonding as described herein, with His192 of the SK4 channel.

In some of any of the embodiments described herein, the compound as described herein is capable of forming hydrogen bonding as described herein, with Arg191 of the SK4 channel. In some of any of the embodiments described herein, the compound as described herein is capable of forming hydrogen bonding with the guanidinium group of Arg191 of the SK4 channel.

In an example, Arg191 features a guanidine group in its side chain which may donate a hydrogen atom due to a weak covalent bond between the amine nitrogen and hydrogen, and the compound of some of the present embodiments features a nitrogen and/or oxygen atom that is capable to bind hydrogen due to its electronegativity.

Similarly, His192 features an imidazole group in its side chain, in which the secondary amine can donate a hydrogen due to a weak bond, and the compound of some of the present embodiments features a nitrogen and/or oxygen atom that is capable to bind, and therefore accept, hydrogen due to its electronegativity.

According to some of any of the embodiments described herein, the at least two functional moieties are spatially arranged such that the compound is capable of forming hydrogen bonds with Arg191 and His192 of the SK4 channel.

According to some of any of the embodiments described herein, the compound comprises at least one functional moiety that is capable of interfering with an interaction between at least one amino acid residue of the calmodulin N-lobe that interacts with the linker S4-S5 of the SK4 channel. Thus, the compound comprises one or more functional moieties that interferes with an interaction between calmodulin and the SK4 channel, and as a result of this interference, the SK4 channel is not activated by the calmodulin. In some of these embodiments, this interference allosterically affects the Ca⁺²-activation of the SK4 channel.

According to some of any of the embodiments described herein, the at least one amino acid residue is selected from Met72 and Met76 of calmodulin.

According to some of any of the embodiments described herein, the at least one functional moiety is spatially arranged such that the compound is capable of sterically hinder the at least one amino acid residue (e.g., Met76) of calmodulin, thereby interfering with an interaction of the calmodulin N-lobe with the linker S4-S5 of the SK4 channel (and thereby allosterically affecting the activation of the SK4 channel).

According to some of any of the embodiments described herein, the at least one functional moiety is spatially arranged such that the compound is capable of forming hydrophobic interactions and/or hydrogen bond interactions with Met 72 of calmodulin. According to some of these embodiments, such interaction with Met72 of calmodulin allosterically affect the activation of the SK4 channel.

According to some of any of the embodiments described herein, the compound comprises at least two functional moieties that are capable of forming hydrogen bonds and/or π-π stacking interactions, the compound being such that when it interacts with the SK4 channel, it is spatially arranged such that the at least two functional moieties that are capable of forming hydrogen bonds and/or π-π stacking interactions are in proximity and orientation that enable formation of hydrogen bonds and/or π-π stacking interactions with Arg191 and His192. That is, the compound is positioned in the channel such that these two functional groups are capable of interacting, as described herein, at least with these two amino acid residues.

According to some of any of the embodiments described herein, the compound further comprises at least one additional functional moiety that is capable of interfering with an interaction between at least one amino acid residue of the calmodulin N-lobe that interacts with the linker S4-S5 of the SK4 channel, the compound being such that when it interacts with the SK4 channel, it is spatially arranged such that the at least two functional moieties that are capable of forming hydrogen bonds and/or π-π stacking interactions are in proximity and orientation that enable formation of hydrogen bonds and/or π-π stacking interactions with Arg191 and His192 and the additional functional moiety is in proximity and orientation that enable steric hindrance of Met76 of calmodulin and/or formation of hydrogen bonds and/or hydrophobic interaction with Met72 of calmodulin. In some embodiments, the compound is positioned in the channel such that two functional groups are capable of interacting, as described herein, at least with the two amino acid residues at the PIP2 binding pocket and one or more functional moieties are capable of interfering with the interaction of calmodulin and the channel, thereby allosterically affecting the activation of the channel by calmodulin.

According to some of any of the embodiments described herein, the compound is capable of allosterically interfering with an interaction of Arg352 of the SK4 channel and calmodulin.

Exemplary compounds that are capable of downregulating SK4 channel in accordance with the embodiments described herein can be collectively represented by Formula I:

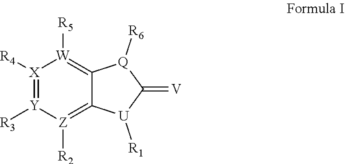

or a pharmaceutically acceptable salt thereof,
wherein:
X, Y, Z and W are each independently carbon or nitrogen, wherein when Z is nitrogen R₂ is absent; when Y is nitrogen, R₃ is absent; when X is nitrogen, R₄ is absent and when W is nitrogen, R₅ is absent;
Q and U are each independently selected from O, S and N, wherein when Q is O or S, R₆ is absent; and when U is O or S, R₁ is absent; and at least one of Q and U being nitrogen (N);
V is O, S or NR₇;
R₁, R₆ and R₇, when present, are each independently selected from hydrogen, alkyl, alkenyl, alkynyl, cycloalkyl, heteroalicyclic, aryl, heteroaryl, hydroxy, alkoxy, thiol, carboxylate, thiocarboxylate, thiohydroxy, carbonyl, carbamate, provided that one of R₁ and R₆ is an alkyl of at least 5 carbon atoms in length; and that when Q and U are each nitrogen, only one of R₁ and R₆ is the alkyl of at least 4, or at least 5, carbon atoms in length; and
R₂, R₃, R₄ and R₅ are each independently selected from hydrogen, alkyl, alkenyl, alkynyl, aryl, heteroaryl, halo, hydroxy, thiol, alkoxy, aryloxy, amine, amide, azo, azide, cyano, carbamyl, hydrazine, ether, ester, carbonyl, sulfonyl, sulfinyl, sulfonamide, thionyl, thioalkoxy, thioalkyl, thioaryl, thioester, thiocarbamide, thiocarbamate.

According to some of any of the embodiments described herein for Formula I, V is O.

According to some of any of the embodiments described herein for Formula I, U is N and R₁ is the alkyl of at least 5 carbon atoms in length.

According to some of any of the embodiments described herein for Formula I, V is O, U is N and R₁ is the alkyl of at least 5 carbon atoms in length.

According to some of any of the embodiments described herein for Formula I, Q is N, and R₆ is hydrogen or an alkyl of up to 3 carbon atoms in length.

According to some of any of the embodiments described herein for Formula I, V is O, Q is N, and R₆ is hydrogen or an alkyl of up to 3 carbon atoms in length.

According to some of any of the embodiments described herein for Formula I, U is N and R₁ is the alkyl of at least 5 carbon atoms in length, and Q is N, and R₆ is hydrogen or an alkyl of up to 3 carbon atoms in length. According to some of these embodiments, V is O.

According to some of any of the embodiments described herein for Formula I, Q is O.

According to some of any of the embodiments described herein for Formula I, U is N and R₁ is the alkyl of at least 5 carbon atoms in length, and Q is O. According to some of these embodiments, V is O.

According to some of any of the embodiments described herein for Formula I, R₂, R₃, R₄ and R₅ are each hydrogen.

According to some of any of the embodiments described herein for Formula I, R₂, R₃, R₄ and R₅ are each independently selected from hydrogen, alkyl, halo, nitro, cyano, alkoxy and thioalkoxy.

According to some of any of the embodiments described herein for Formula I, X, Y, Z and W are each carbon.

According to some of any of the embodiments described herein for Formula I, X, Y, Z and W are each carbon, and R₂, R₃, R₄ and R₅ are each hydrogen.

According to some of any of the embodiments described herein for Formula I, X, Y, Z and W are each carbon; R₂, R₃, R₄ and R₅ are each hydrogen; and V is O.

According to some of any of the embodiments described herein for Formula I, X, Y, Z and W are each carbon; and V is O.

According to some of any of the embodiments described herein for Formula I, R₂, R₃, R₄ and R₅ are each hydrogen; and V is O.

According to some of any of the embodiments described herein for Formula I, X, Y, Z and W are each carbon; R₂, R₃, R₄ and R₅ are each hydrogen; V is O; U is N; R₁ is the alkyl of at least carbon atoms in length; and Q is O.

According to some of any of the embodiments described herein for Formula I, X, Y, Z and W are each carbon; V is O; U is N; R₁ is the alkyl of at least 5 carbon atoms in length; and Q is O.

According to some of any of the embodiments described herein for Formula I, R₂, R₃, R₄ and R₅ are each hydrogen; V is O; U is N; R₁ is the alkyl of at least 5 carbon atoms in length; and Q is O.

According to some of any of the embodiments described herein for Formula I, V is O, U is N and R₁ is the alkyl of at least 5 carbon atoms in length.

According to some of any of the embodiments described herein for Formula I, X, Y, Z and W are each carbon; R₂, R₃, R₄ and R₅ are each hydrogen; V is O; U is N; R₁ is the alkyl of at least carbon atoms in length; Q is N; and R₆ is hydrogen or an alkyl of up to 3 carbon atoms in length.

According to some of any of the embodiments described herein for Formula I, X, Y, Z and W are each carbon; V is O; U is N; R₁ is the alkyl of at least 5 carbon atoms in length; Q is N; and R₆ is hydrogen or an alkyl of up to 3 carbon atoms in length.

According to some of any of the embodiments described herein for Formula I, R₂, R₃, R₄ and R₅ are each hydrogen; V is O; U is N; R₁ is the alkyl of at least 5 carbon atoms in length; Q is N; and R₆ is hydrogen or an alkyl of up to 3 carbon atoms in length.

According to some of any of the embodiments described herein, the compounds can be collectively represented by Formula II:

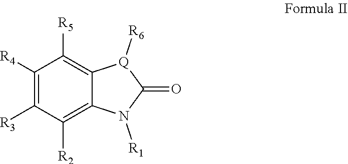

or a pharmaceutically acceptable salt thereof,
wherein R₁-R₆ and Q are as described in any of the embodiments described herein for Formula I, and any combination thereof.

According to some of any of the embodiments described herein, the compounds are collectively represented by Formula III:

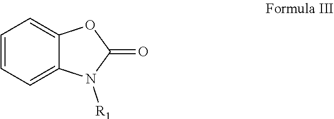

or a pharmaceutically acceptable salt thereof,
wherein R₁ is an alkyl of at least 5 carbon atoms in length.

According to some of any of the embodiments described herein, the compounds are collectively represented by Formula IV:

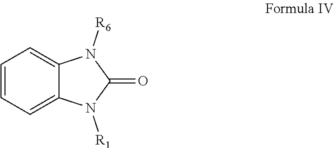

or a pharmaceutically acceptable salt thereof,
wherein R₁ and R₆ are each independently selected from hydrogen, alkyl, alkenyl, alkynyl, cycloalkyl, heteroalicyclic, aryl, heteroaryl, hydroxy, alkoxy, thiol, carboxylate, thiocarboxylate, thiohydroxy, carbonyl, carbamate, provided that one of R₁ and R₆ is an alkyl of at least 4, or at least 5 carbon atoms in length; and that only one of R₁ and R₆ is an alkyl of at least 4, or at least 5, carbon atoms in length.

Herein throughout, an alkyl of at least 4 or at least 5 carbon atoms in length describes a linear or branched alkyl which features a chain of at least 4 or at least 5 carbon atoms, that is, it features a saturated linear chain of 4, 5, 6, 7, 8, 9, 10, 11, 12, 13, 14, 15, and so forth, carbon atoms, each can independently be substituted or unsubstituted.

According to some embodiments, the alkyl of at least 4 or at least 5 carbon atoms in length has a saturated linear chain of from 4 to 30, or from 4 to 25, or from 4 to 20, or from 4 to 15, or from 4 to 12, or from 4 to 10, or from 5 to 30, or from 5 to 25, or from 5 to 20, or from 5 to 15, or from 5 to 12, or from 5 to 10, carbon atoms, including any intermediate values and subranges therebetween.

According to some of any of the embodiments described herein, the alkyl of at least 4 or 5 carbon atoms in length as described herein is unsubstituted.

Herein throughout, an alkyl of up to 3 carbon atoms in length describes a linear or branched alkyl which features a chain of 1, 2 or 3 carbon atoms, that is, it is a substituted or unsubstituted methyl, ethyl or propyl.

According to some of any of the embodiments described herein, the alkyl of up to 3 carbon atoms in length as described herein is unsubstituted. According to exemplary embodiments, it is a methyl.

According to some of any of the embodiments described herein, the compound as described herein in any of the respective embodiments (e.g., a compound that features functional groups that are spatially arranged to interact with the domains as described herein, and/or a compound of Formula I, II, III or IV) is for use in treating a medical condition associated with overexpression and/or overactivity of SK4 channel.

The phrase “overexpression and/or overactivity of SK4 channel”, as used in any of the embodiments described herein, refers to an elevated abnormal level of expression and/or activity of SK4 channel in a given cell.

In some of any of the embodiments described herein, downregulating the SK4 channel as described in any of the respective embodiments comprises administering to a subject in need thereof (e.g., a subject having or suspected as having abnormal expression and/or activity of SK4 channel, or a subject having or suspected as having a medical conditions associated with abnormal expression and/or activity of SK4 channel) an effective amount (e.g., a therapeutically effective amount) of a compound, as defined herein in any of the respective embodiments.

In the context of any of the embodiments described herein, an effective amount is an amount sufficient to reduce or inhibit a function of a SK4 channel, as defined herein.

According to some of any of the embodiments described herein, the medical condition is associated with cardiac arrhythmia.

According to some of any of the embodiments described herein, the medical condition is an atrial arrhythmia.

According to some of any of the embodiments described herein, the medical condition is a ventricular arrhythmia.

According to some of any of the embodiments described herein, the medical condition is catecholaminergic polymorphic ventricular tachycardia (CPVT).

According to some of any of the embodiments described herein, medical conditions associated with SK4 channel activity and/or expression, or which can benefit from downregulating an activity and/or expression of SK4 channel, include medical condition in which inducing bradycardia (e.g., slowing a heart rate) is desirable or beneficial in a subject in need thereof.

The term “bradycardia”, which is also known as “bradyarrhythmia”, as used herein and in the art, describes a slow heart rate in a subject compared to a normal, average, heart rate of a healthy subject of the same age and species, or compared to a heart rate associated with a subject's medical condition.

Bradycardia can be determined, for example, by electrocardiography (ECG).

The term “bradycardia” encompasses atrioventricular nodal bradycardia (AV junction rhythm), which usually appears on an ECG with a normal QRS complex accompanied with an inverted P wave either before, during, or after the QRS complex, and ventricular bradycardia, which is manifested by a slow heart rate (e.g., of less than 50 BPM in human adult), which usually appears as imbalanced relationship between P waves and QRS complexes in ECG. By “inducing bradycardia” are encompassed slowing a heart rate of a subject (e.g., reducing the heart rate of the subject by, for example, at least 5% or at least 10% or at least 20% or at least 30%, or at least 40% or at least 50%, compared to the heart rate of the same subject before treatment), and/or regulating an increased heart rate such that the heart rate of the subject is within the acceptable range of a healthy subject (e.g., of the same age and other parameters), and/or decreasing the sinus rate (by, for example, at least 5% or at least 10% or at least 20% or at least 30%, or at least 40% or at least 50%, compared to the sinus rate of the same subject before treatment) and/or elongating/prolonging the PR interval (by, for example, at least 5% or at least 10% or at least 20% or at least 30%, or at least 40% or at least 50%, compared to the PR interval of the same subject before treatment).

A “Sinus rate”, which is also known and referred to in the art as “sinus rhythm”, can be defined by the morphology of P waves in ECG.

The term “PR interval” as used herein, which is also known and referred to in the art as “PQ interval”, is defined as the period that extends from the beginning of the P wave (the onset of atrial depolarization) until the beginning of the QRS complex (the onset of ventricular depolarization), in ECG.

Subjects in need of induction of bradycardia include, for example, subjects suffering from a medical condition in which inducing bradycardia (i.e., slowing a heart rate) is desirable or beneficial. Bradycardic effect and slowed atrioventricular node conduction exhibited by downregulating the SK4 channel can be desirable or beneficial for preventing ventricular tachycardia by prolonging the refractory period, as an alternative to, e.g., the currently used β1-adrenergic and Ca²⁺ channel blockers, as well as in treating other cardiac arrhythmias of different etiologies, non-arrhythmic cardiovascular disorders (cardiac diseases), ventricular tachyarrhythmias in CPVT and possibly in other arrhythmic pathologies of different etiologies such as the long QT syndrome.

In some embodiments, the medical condition is a cardiac disease or disorder, and in some embodiments, the medical condition is a cardiac arrhythmia disease or disorder.

In some embodiments, the method according any of the respective embodiments can be used to treat cardiac disorders characterized by abnormal cardiac rhythm, such as, for example, cardiac arrhythmia.

In some embodiments, the medical condition is associated with cardiac arrhythmia.

In some embodiments, the medical condition is such that requires a procedure which is advantageously performed while slowing a heart rate of the subject, for example, a surgery that involves interception of an organ or tissue of the cardiovascular system or any other operation of the cardiovascular system. An example is an open heart surgery.

Any other cardiac as well as non-cardiac diseases or disorders or medical conditions in which slowing a heart rate is beneficial are contemplated.

As used herein the phrase “cardiac arrhythmia” refers to a variation from the normal rhythm of the heart rate, for example, tachycardia.

The cardiac arrhythmia can be a ventricular arrhythmia, an atrial arrhythmia, a junctional arrhythmia and a heart block.

Medical conditions associated with atrial arrhythmia include, but are not limited to, Premature atrial contractions (PACs), Wandering atrial pacemaker, Atrial tachycardia, Multifocal atrial tachycardia, Supraventricular tachycardia (SVT), Atrial flutter, and Atrial fibrillation (Afib).

Medical conditions associated with junctional arrhythmia include, but are not limited to, AV nodal reentrant tachycardia, Junctional rhythm, Junctional tachycardia, and Premature junctional contraction

Medical conditions associated with ventricular arrhythmia include, but are not limited to, Premature ventricular contractions (PVCs), sometimes called ventricular extra beats (VEBs), Premature ventricular beats occurring after every normal beat are termed “ventricular bigeminy”, Accelerated idioventricular rhythm, Monomorphic ventricular tachycardia, Polymorphic ventricular tachycardia, Ventricular fibrillation, and Torsades de pointes.

Medical conditions associated with heart block include, but are not limited to, AV heart blocks, which arise from pathology at the atrioventricular node, including First degree heart block, which manifests as PR prolongation, Second degree heart block, including Type 1 Second degree heart block, also known as Mobitz I or Wenckebach, and Type 2 Second degree heart block, also known as Mobitz II, and Third degree heart block, also known as complete heart block.

Exemplary medical conditions associated with cardiac arrhythmia include, but are not limited to, atrial fibrillation, ventricular fibrillation, conduction disorders, premature contraction, and tachycardia.

Conduction disorders collectively encompass abnormal or irregular progression of electrical pulses through the heart, which cause a change in the heart rhythm. Conductions disorders are not necessarily associated with arrhythmia but sometimes are the cause of arrhythmia. Exemplary conductions disorders include, but are not limited to, Bundle Branch Block, heart block, including first-, second- and third-degree heart block, and long Q-T syndrome.

Premature contraction includes premature atrial contractions and premature ventricular contractions.

Additional exemplary medical conditions associated with arrhythmia include Adams-Stokes Disease (also called Stokes-Adams or Morgagni), atrial flutter, which is usually found in patients with: Heart failure, Previous heart attack, Valve abnormalities or congenital defects, High blood pressure, Recent surgery, Thyroid dysfunction, Alcoholism (especially binge drinking), Chronic lung disease, Acute (serious) illness, Diabetes, after open-heart surgery (bypass surgery), or atrial fibrillation; Sick Sinus syndrome; sinus arrhythmia and Wolff-Parkinson-White (WPW) syndrome.

In some of any of the embodiments described herein, the cardiac disease or disorder is associated with tachycardia.

The term “tachycardia”, which is also known as “tachyarrhythmia”, as used herein and in the art, describes a fast heart rate in a subject compared to a normal, average, heart rate of a healthy subject of the same age and species, or compared to a heart rate associated with a subject's medical condition.

Tachycardia can be determined, for example, by electrocardiography (ECG), and encompasses a wide range of conditions, as listed herein throughout.

In some embodiments, the tachycardia encompasses atrial and Supraventricular tachycardia (SVT), including paroxysmal atrial tachycardia (PAT) or paroxysmal supraventricular tachycardia (PSVT); Sinus tachycardia, which can be associated with disorders of that heart which interfere with the normal conduction system of the heart, including, but not limited to, Lack of oxygen to areas of the heart due to lack of coronary artery blood flow, Cardiomyopathy in which the structure of the heart becomes distorted, Medications, Illicit drugs such as cocaine, and Sarcoidosis (an inflammatory disease affecting skin or other body tissues).

In some embodiments, the tachycardia is a ventricular tachycardia, a supraventricular tachycardia, atrial fibrillation, AV nodal reentrant tachycardia (AVNRT), or an AV reentrant tachycardia (AVRT).

In some embodiments, the cardiac disease or disorder is CPVT, as described herein and in the art.

In some embodiments, the cardiac disease or disorder is a long QT syndrome.

In some of any of the embodiments described herein, the medical condition or disorder is myocardial infarction (MI).

As used herein the phrase “myocardial infarction” refers to the loss of cardiac myocytes or myocardial cell death caused by prolonged ischemia (i.e., a condition in which insufficient flow of oxygenated blood reaches the tissues and organs).

The myocardial infarction can be an acute coronary syndrome (ACS), acute myocardial infarction (AMI), coronary artery disease (CAD), congestive heart failure (CHF), cardiomyopathy (CM), cardiothoracic (CT), percutaneous coronary intervention (PCI), pulmonary embolism (PE), or ST-segment elevation myocardial infarction (STEMI).

According to some of any of the embodiments described herein, the medical condition is fibrosis.

As used herein the term “fibrosis” refers to the formation of a scar tissue as a result of injury or inflammation. Fibrosis can occur in various organs and tissues throughout the body, and it can lead to a number of medical conditions.

Examples of medical conditions that involve fibrosis include, but are not limited to, cardiac fibrosis, liver fibrosis, pancreatic fibrosis, scarring of the vocal cords, fibrosis of the vocal cord mucosa, laryngeal fibrosis, pulmonary fibrosis, idiopathic pulmonary fibrosis, cystic fibrosis, myelofibrosis, retroperitoneal fibrosis, nephrogenic systemic fibrosis, kidney fibrosis, keloids, Dupuytren's contracture, dermatofibrosis lenticularis disseminate, morphea and scleroderma.

According to some of any of the embodiments described herein, the medical condition is cardiac fibrosis.

As used herein the term “cardiac fibrosis” refers to an excess of deposited extracellular matrix (ECM) by cardiac fibroblasts as a result of injury or inflammation, e.g., a complication of various cardiovascular conditions, such as heart failure and hypertension. Cardiac fibrosis impairs the heart physically and electrically, and can lead to a number of medical conditions, e.g., cardiac dysfunction, heart failure.

The subject to be treated according to some of any of the embodiments of the present invention can be a mammal, preferably a human being, including a neonatal, a baby, an infant, and an adult.

In some embodiments, the subject is afflicted by, or suffers from, any of the medical conditions as described herein.

Tachycardia and bradycardia are defined in a subject in accordance with acceptable heart rates defined as normal in accordance with a subject's age.

Any of the compounds as described herein can be administered to an organism per se, or in a pharmaceutical composition where it is mixed with suitable carriers or excipients.

According to some of any of the embodiments described herein, the subject is a human subject.

According to some of any of the embodiments described herein, the subject is a post-natal (e.g., adult) human subject.

According to some of any of the embodiments described herein, the compound forms a part of a pharmaceutical composition which further comprises a carrier.

According to an aspect of some embodiments of the present invention there is provided a compound represented by Formula I, II, III or IV as described herein in any of the respective embodiments and any combination thereof.

According to an aspect of some embodiments of the present invention there is provided a pharmaceutical composition comprising the compound as described herein in any of the respective embodiments and any combination thereof, and pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compound accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences”, Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (any of the compounds described herein) effective to prevent, alleviate or ameliorate symptoms of a disorder (e.g., associated with SK4 channel as described herein) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

For any of the methods, uses and compositions as described herein, the compound can be utilized (e.g., co-administered) or formulated with an additional active agent that is usable in treating the medical condition and/or in downregulating an activity of SK4 channel.

The findings onto which some of the present embodiments are based can be beneficially utilized for identifying compounds capable of downregulating an activity of an SK4 channel.

According to an aspect of some embodiments of the present invention there is provided a method of identifying a candidate compound that is capable of downregulating an activity of SK4 channel. The method according to these embodiments is generally effected by computationally docking a library of compounds into a calmodulin-PIP2 binding domain of an SK4 channel; and determining if a compound is arranged such that it interacts with one or more amino acid residues in the binding domain, as described herein in any of the respective embodiments and any combination thereof.

According to some of any of the embodiments of this aspect of the present invention, the at least one of the amino acid residues is selected from Arg191 and His192 of the SK4 channel.

According to some of any of the embodiments of this aspect of the present invention, the at least one of the amino acid residues is selected from Arg191 and His192 of the SK4 channel, and Met76 and Met72 of calmodulin.

A compound that is arranged such that it interacts with the one or more amino acid residues in the binding domain is identified as a candidate compound for downregulating an activity of SK4 channel.

Determining an arrangement of the compound can be performed using any available method and/or system for computational docking. Examples include molecular docking programs and/or algorithms such as AutoDock, DOCK, FlexAID, LeDock, rDock, Glide, SEED and PLANTS. Following docking, molecular dynamics (MD) simulations can be used to optimize the simulated docked complex and to provide detailed information about the structures and specific interactions between the materials at the atomic and molecular level. Examples include molecular dynamics (MD) simulations such as GROMACS (GROningen MAchine for Chemical Simulations), AMBER (Assisted Model Building with Energy Refinement), CHARMM (Chemistry at Harvard Macromolecular Mechanics), LAMMPS (Large-scale Atomic/Molecular Massively Parallel Simulator), and NAMD (NAnoscale Molecular Dynamics).

Once candidate compounds are identified by computational docking, these compounds can be further tested in in vivo, ex vivo and/or in vivo assays, to evaluate their effect on the activity of the target SK4 channel, to thereby identify lead compounds.

The identified compounds can be subjected to further studies to determine their therapeutic index and other pharmacological parameters so as to evaluate their suitability as potential drugs for treating any of the medical conditions as described herein.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ group or a —N⁺R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group refers to a —N═N⁺═N⁻ group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R'S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R′″ are as defined herein.

A “guanidinyl” group refers to an —RaNC(═NRd)—NRbRc group, where each of Ra, Rb, Rc and Rd can be as defined herein for R′ and R″.

A “guanyl” or “guanine” group refers to an RaRbNC(═NRd)— group, where Ra, Rb and Rd are as defined herein.

According to some of any of the embodiments described herein, any of the compounds prepared or provided according to the present embodiments can be in a form of a pharmaceutically acceptable salt thereof.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, and/or to improve its stability, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., an amine-containing group such as amine and/or guanidyl and/or guanyl) group of the compound which is in a positively charged form (e.g., wherein the basic group is protonated), in combination with at least one counter-ion, derived from the selected base, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

Chemicals: all chemicals were purchased from Tzamal D-Chem Laboratories Ltd. Petah-Tikva, Israel. All solvents were purchased from BioLab Ltd. (Israel). Novel synthesized compounds were designed and practiced as described below herein (see Example 2).

Constructs: For CHO cell transfection, the following were cloned: human SK1, rat SK2, human SK3, and rat calmodulin into the pcDNA3 vector, human SK4 into pEGFP-C1 vector, and PIP4,5-kinase into an IRES-dsRed plasmid. The mutations were introduced using the PCR-based QuikChange™ site-directed mutagenesis (Stratagene®) and were verified by full sequencing of the entire plasmid vector.

Drugs: Poly L-Lysine 50 μg/ml (poly-L-lysine hydrochloride, MW=8,200 Da (Alamanda Polymers Inc.); PI(4,5)P2 diC8 (dioctanoyl, phosphatidylinositol 4,5 bisphosphate; echelon biosciences); Tram-34 (Alomone Labs™); Carbachol (carbamylcholine chloride; Sigma-Aldrich@).

Cell Culture and Transfection: Chinese hamster ovary (CHO) cells were grown in Dulbecco's modified Eagle's medium supplemented with 2 millimolar (mM) glutamine, 10% fetal calf serum, and antibiotics. In brief, 40,000 cells seeded on poly-L-lysine-coated glass coverslips (13 millimeter (mm) in diameter) in a 24-multiwell plate were transfected with 0.5 microgram (μg) pEGFP-SK4/0.5 μg mutant SK4, 1.2 μg dsRed-PIP4,5-kinase or with 1 μg SK1/SK2/SK3 together with pIRES-CD8 (0.3 μg) as a marker for transfection. Transfection was performed using TransIT-LT1 Transfection Reagent (Mirus Bio) according to the manufacturer's protocol. For electrophysiology, transfected cells were visualized approximately 40 hours after transfection with a Zeiss Axiovert 35 inverted florescence microscope.

### Electrophysiology Methods:

Whole-cell patch-clamp recording: Voltage-clamp recordings were performed using the whole-cell configuration of the patch-clamp technique. Signals were amplified using an Axopatch 700B patch-clamp amplifier (Molecular Devices), sampled at 5 kHz and filtered at 2.4 kHz via a four pole Bessel low pass filter. Data were acquired using pClamp™ 10.5 software in conjunction with an Axon™ DigiData® 1440A interface (Molecular Devices). The patch pipettes were pulled from borosilicate glass (Harvard Apparatus) with a resistance of 3-5 megaohms (MΩ). The intracellular pipette solution contained 130 mM KCl, 5 mM EGTA, 10 mM HEPES, pH 7.3 (adjusted with KOH), and CaCl₂ calculated for a final concentration of 1 μM free-Ca²⁺, by MAXCHELATOR (WEBMAXC STANDARD) software, with sucrose added to adjust osmolarity to 290 mosM. The external solution (310 mosM) contained 140 mM NaCl, 4 mM KCl, 1.8 mM CaCl₂, 1.2 mM MgCl₂, 11 mM glucose, and 5.5 mM HEPES adjusted with NaOH to pH 7.3. CHO cells were held at −90 millivolt (mV) and SK4 K⁺ currents were activated using a voltage ramp protocol from −100 mV to +60 mV for 150 milliseconds. Electrophysiological data analysis was performed using the Clampfit program (pClamp™ 10.5; Molecular Devices).

Inside-out macro-patch recording: To measure the Ca²⁺ concentration dependence for SK4 channel activation, CHO cells were co-transfected with plasmids encoding WT SK4 (1 μg) and WT CaM (1 μg) using the Lipofectamine reagent at a ratio of 1:2. The cells were transfected 48 hours prior to recordings. The patch pipettes were pulled and fire-polished with a MF-900 micro-forge (Narashige) to reach an internal diameter of 3-5 μM. The resistance of the patch electrodes ranged from 2-3 MΩ. The pipette solution contained 135 mM KCl, 1 mM MgSO₄, 0.91 mM CaCl₂, and 10 mM HEPES at pH 7.3. The bath solution contained 135 mM KCl, 5 mM EGTA, and 10 mM HEPES at pH 7.3. EGTA was used to titer the different Ca²⁺ concentration solutions, calculated using the software by C. Patton of Stanford University (http://maxchelator(dot)stanford(dot)edu/). Currents were recorded by 10 repetitive 1 second duration voltage ramps from −100 mV to +100 mV from a holding potential of 0 mV. The current amplitudes in response to increasing Ca²⁺ concentrations were normalized to those obtained at a saturating Ca²⁺ concentration (3 μM). Furthermore, to determine the Ca²⁺ concentration dependence for activation in the presence of a tested compound, such as the exemplary compound BA6b9 (10 μM), the current amplitudes were normalized to those obtained at large maximal Ca²⁺ concentration of 10 μM. For PIP2-dependent activation, PIP2 affinity to the SK4 channel was also examined in the inside-out configuration. The dose-response curve for PIP2 was measured with increasing concentrations of diC8-PIP2 in the presence of 1 μM Ca²⁺. The diC8-PIP2 effect on the channel current was measured after complete depletion of the native PIP2 using sonicated poly-L-lysine (PLL) 50 μg/ml and a subsequent 2-minute washout. The current amplitudes in response to increasing diC8-PIP2 concentrations were normalized to those obtained at maximal diC8-PIP2 concentration of 10 μM in the presence of 1 μM Ca²⁺. Apparent EC₅₀ values for Ca²⁺ or diC8-PIP2 were determined by fitting the data points to a standard dose-response curve Y=100/(1+10^{(log EC(50)-X)*HillSlope}).

Electrophysiological data analyses: Data analysis was performed using the Clampfit program (pClamp™ 10.5; Axon Instruments), Microsoft Excel™ (Microsoft®, Redmond, WA), and Prism

9.0 (GraphPad Software, Inc., San Diego, CA). Leak subtraction was performed off-line, using the Clampfit program of the pClamp™ 10.5 software. All data were expressed as mean±S.E.M. Statistical differences were assessed by paired, unpaired two-tailed Student's t-test or one-way ANOVA, as indicated in figure legends.

### Ex-Vivo Experiments:

Animals: Adult male Sprague-Dawley rats (n=56, 200-250 grams) and Guinea pigs (n=19, 400-700 grams) obtained from Envigo Laboratories (Jerusalem, Israel) were bred and grown in Ben-Gurion University of the Negev, Israel. Experiments in rats and guinea pigs were approved by the institutional ethics committee of Ben-Gurion University of the Negev, Israel (Protocol No. IL-24-05-2020(D) and were carried out in strict accordance with the Guide for the Care and Use of Laboratory Animals of the National Institutes of Health. The animals were kept under standardized conditions: 12:12 light: dark cycles at 20-24° C. and 30-70% relative humidity.

Animals were free-fed autoclaved rodent chow and had free access to reverse osmosis filtered water. Hearts were excised from the animals under deep pentobarbital anesthesia.

Perfused isolated hearts—Langendorff method: The isolated heart experiment setup was performed as described in Etzion et al., Am J Physiol Heart Circ Physiol (2008) 295, H1460-1469; and Mor et al., J Pharmacol Exp Ther (2013) 344, 59-67.]. Briefly, each animal was anesthetized with 2% isoflurane and an intraperitoneal (IP) injection of pentobarbital (60 mg/kg), followed by IP injection of Heparin (1000 U/kg). The heart was rapidly excised and placed into ice-cold Tyrode's buffer solution (consisted of 140 mM NaCl, 5.4 mM KCl, 0.5 mM MgCl₂, 2.5 mM CaCl₂, 0.39 mM NaH₂PO₄, 10 mM HEPES and 11 mM glucose, and titrated to pH 7.4 with NaOH). The aorta was cannulated and connected to a pre-heated (37° C.) and oxygenated perfusion system with Tyrode's solution while perfusion pressure was maintained at about 70 mmHg throughout the experiment. Prior to the experiment, the heart was left in Tyrode's solution to stabilize for 20 minutes for hemodynamic measurements, the left atrial appendage was excised, and a collapsed latex balloon was inserted into the left ventricle (LV) through the mitral valve. Once positioned, the balloon was filled with double distilled water reaching an end-diastolic pressure of 10-15 mmHg. Coronary perfusion pressure and LV pressure were recorded by a pressure amplifier (ETH-256C amplifier and B-100 probes, iWorx, NH, USA). Electrophysiological signals were recorded from the high right atrium (HRA) via a miniature quadripolar hook electrode (for simultaneous pacing and recording) and from the LV via a bipolar hook electrode. Electrical signals were filtered (1-2 kHz) and recorded by two voltage amplifiers (Model 3000, A-M Systems, Carlsberg, WA, USA). Signals were interfaced with a PC using an A/D converter (PCI-6024E, National Instruments, Austin TX, USA) and a custom-designed program developed with LabVIEW programing language (National Instruments, Austin, TX, USA) to control signal acquisition, data saving and off-line analysis. Measured physiological parameters during the experiment included perfusion flow (ml/minute) as a marker for coronary perfusion pressure, LV developed pressure, dP/dt±(representing the rate of rise of left ventricular pressure during isovolumetric contraction as a marker for LV contractility), heart rate, PR, and RR intervals. Tram-34 and BA6b9 were applied for a 20-minutes incubation period and physiological parameters were recorded.

Effective refractory period measurements: Using both custom-made quadripolar electrode inserted on the HRA and two bipolar electrodes (recording and pacing) on the LV, allowed measurement of the effective refractory period (ERP) at the atrial level (atrial effective refractory period, AERP), atrioventricular level (AVERP), and ventricular level (VERP). For ERP measurement, a programmed S1S2 stimulation protocol was performed using double diastolic threshold intensity. The protocol consisted of ten S1-S2 intervals of 150 milliseconds (ms) followed by an S1-S2 interval that was reduced by 1 ms each time, until pacing capture failed three consecutive times. AERP, AVERP, and VERP were measured and recorded prior and after exposure to the used inhibitor, either Tram-34 or BA6b9.

Atrial Fibrillation induction in Sprague-Dawley rats: For evaluation of Tram-34 and BA6b9 on atrial fibrillation (AF) induction, a custom-made quadripolar electrode was inserted for simultaneous recording and pacing on the HRA. To increase susceptibility for reentry and atrial tachyarrhythmia, the cholinergic agonist carbachol was used. First, baseline AERP was measured under normal physiological conditions, and the measurement was repeated upon exposure to 0.3 μM carbachol, 0.3 μM carbachol+10 μM Tram-34 or 0.3 μM carbachol+10 μM BA6b9. Then, for AF induction, burst pacing was applied to the HRA under increasing pacing thresholds (2×, 3×, 4×, 5×, and 6× threshold). This protocol included two consecutive 5 second bursts at a cycle length of 20 milliseconds for each threshold. This protocol was tested under three conditions: normal physiological conditions, 0.3 μM carbachol, 0.3 μM carbachol+10 μM Tram-34, or 0.3 μM carbachol+10 μM BA6b9. To determine AF susceptibility under each condition, an AFIS (AF induction score) ranking system was created, relying on the level of threshold intensity when AF induction was successful. Lower AF induction threshold means higher AFIS. The ranking was as follows: induction at 2×-diastolic threshold received the highest score of 5, at 3× the received score was 4, at 4× the received score was 3, at 5× the received score was 2, at 6× the received score was 1, and if no induction occurred it scored zero. For AF sustainability quantification, sustained AF was determined as lasting ≥5 minutes. Non-sustained AF received a score of zero and sustained AF received a score of 1.

### In-Vivo Experiments:

In a rat model of heart failure (HF) post-myocardial infarction (MI) (with ejection fraction EF<40%), daily treatment with the exemplary compound BA6b9 (20 mg/kg) were performed for 3 weeks starting one week post-MI, and were compared with similar treatment with vehicle as control. Experimental setup and details are described, for example, in Murninkas et al. Am J Physiol-Heart Circul Physiol. (2021), 320, H713-H24.

Following electrode implantation and animal recovery, baseline EP (electrophysiological) measurements were performed followed by random division of the animals to treatment with the exemplary compound vs. treatment with vehicle.

Masson-trichrome staining: staining was performed according to a standard procedure, as described, for example, in N. Foot, Stain Technology (2009), Volume 8, 1933—Issue 3, 101-110.

### Computational Methods:

System preparation: Molecular Dynamics were performed for the human SK4/calmodulin complex cryo-EM structure in its open conformation (PDB code: 6CNN). Since the cryo-EM map was recorded in the absence of PIP2, PIP2 was docked to the interface of calmodulin and the SK4 proximal C-terminus with calmodulin T79 serving as an anchor and using the Glide docking algorithm, as implemented in Maestro version 11.2 (Schrödinger). Next, the Orientations of Proteins in Membranes (OPM) and Membrane Builder in CHARMM-GUI web-servers were used to build a system of SK4/calmodulin/PIP2 in a POPC lipid bilayer. Then the system was solvated in TIP3P water molecules to form a 162×162×142 angstrom (Å) simulation box. Finally, potassium and chloride ions were added to the water phase in order to neutralize the system and to obtain a salt concentration of 0.15 M.

Molecular Dynamics (MD) Simulation: MD simulation was performed with Gromacs 2018.2 with CHARMM36 force field. The simulation was conducted using periodic boundary conditions (PBC) with particle-mesh Ewald (PME) electrostatics with 12 Å cutoff for long range interactions. The simulation was composed of three steps: First, energy minimization with the steepest descent minimization algorithm; second, six equilibration steps with restraints that were applied on protein and membrane atoms. The restrains were gradually reduced to zero during these steps; finally, production simulation for 200 nanoseconds (ns) with a constant temperature of 310 K and a constant pressure of 1 atm (under Nos6-Hoover and Parrinello-Rahman coupling algorithms, respectively), and with an integration time step of 2 femtosecond (fs).

Molecular Data Analysis: The resulting trajectories were visually inspected using VMD 1.9.3 software. The stability of the resulting trajectories was tested based on the root mean square deviation (RMSD), which was calculated using the rms utility of Gromacs 2018.2 package. Next, in order to find the most prevalent conformations, the simulation trajectory was clustered using the Gromos clustering algorithm and a cut-off of 0.2 nm. Based on the clustering analysis, centers of four largest clusters which cover 90% of the conformational space of the trajectory simulation, were picked for further calculations.

Docking of small molecules: The tested/designed molecules were docked into the protein structures. Prior to docking, the protein structures were prepared using the Protein Preparation Wizard in Schrödinger Maestro. Docking calculations were preformed using Glide, as implemented in Maestro version 11.2 (Schrödinger). Glide's grid was centered on Arginine 180 (R180), with box size of 10 Å. The docking calculations were performed in the presence of calmodulin, as well as in its absence, in order to identify molecules that may interfere calmodulin binding. Next, Prime MM-GBSA module in Maestro version 11.2 (Schrödinger) was used to optimize the complexes obtained from docking calculations in the presence of implicit membrane.

### Synthetic and Analytical Instrumentation:

Nuclear magnetic resonance (NMR): Proton and carbon NMR spectra were obtained on either a 500 MHz spectrometer or a 400 MHz spectrometer and are reported in ppm (δ). Dimethylsulfoxide-d₆ (DMSO-d₆) was used as solvent.

Microwave irradiation: Microwave irradiation was performed using CEM Discover SP machine.

### Example 1

### The Role of PIP2 as a Component in SK4 K⁺ Channel Gating

PIP2, phosphatidylinositol 4,5-bisphosphate, is a lipid abundant in animal and plant cells, where it is known to be involved in various signaling pathways.

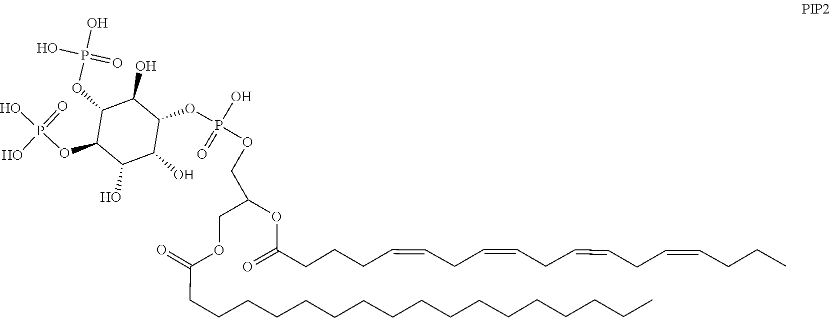 [SMILES: CCC/C=C\C/C=C\C/C=C\C/C=C\CCCC(=O)OC(COC(=O)CCCCCCCCCCCCCCCCC)COP(=O)(O)O[C@@H]1[C@H](O)[C@H](O)[C@@H](OP(=O)(O)O)[C@H](OP(=O)(O)O)[C@H]1O]

One of its roles is as human CaM-SK activator in SK1-SK3 K⁺ channels. The PIP2-binding site was determined in SK2 channels and specific sequence from this region were found to be conserved in SK1 and SK3 as well [Zhang et al., Sci Adv (2015) 1, e1500008; Zhang et al., Nat Commun (2012) 3, 1021; Zhang et al., Nat Chem Biol (2014) 10, 753-759].

The PIP2 molecule bears a net negative charge at neutral pH that allows it to engage in electrostatic interactions with positively charged regions in various proteins [Lee et al., Science (2018) 360, 508].

In order to study the mechanism of SK4 channel activity, the role of PIP2 was examined.

The calcium dependence of the human SK4 K⁺ channel in transfected CHO cells was characterized using inside-out macro-patches. The expressed currents were recorded in response to voltage ramps (−100 millivolt (mV) to +100 mV for 1 seconds) and by increasing the free-Ca²⁺ concentrations applied to the internal face of the membrane. The obtained data is presented in FIG. 1A. Currents were normalized to the maximum response evoked by 1 μM free-Ca²⁺, plotted as a function of Ca²⁺ concentrations and data points were fitted to a Hill equation, as presented in FIG. 4A (see, ‘control’ labeled plot), yielding an EC₅₀ of 65 nM, similar to a previously reported value [Zhang et al., Nat Commun (2012) 3, 1021].

To examine whether PIP2 is involved in the modulation of the SK4 channel, channel trafficking to the plasma membrane was increased by co-transfecting CHO cells with plasmid DNAs encoding for the channel and for wild type (WT) CaM. The resulting currents were recorded from inside-out macro-patches, where the intracellular solution contained a saturating concentration of 1 μM free-Ca²⁺, as shown in FIG. 1B (see, black plot). As shown in FIG. 1B and FIG. 1C, application of poly-L-lysine (PLL) (50 μg/ml), a known PIP2 scavenger, caused a rapid decrease in SK4 K⁺ currents, with 68% inhibition after 1 minute of PLL application, which could not be recovered by increases in intracellular free-Ca²⁺ concentrations.

In the presence of the water-soluble synthetic PIP2 derivative diC8-PIP2, dose-dependent activation of the SK4 K⁺ currents at 1 μM internal free-Ca²⁺ was observed, as can be seen in FIG. 1D. Normalizing the currents to the maximal activating diC8-PIP2 concentration of 10 μM and fitting the data, yielded an EC₅₀ of 154 nM, as can be seen in FIG. 1E. This is a higher value compared to the one obtained in WT SK2 channels (1.9 μM, according to Zhang et al., Nat Chem Biol (2014) 10, 753-759), indicating higher affinity of PIP2 to WT SK4 channel compared to WT SK2 channel.

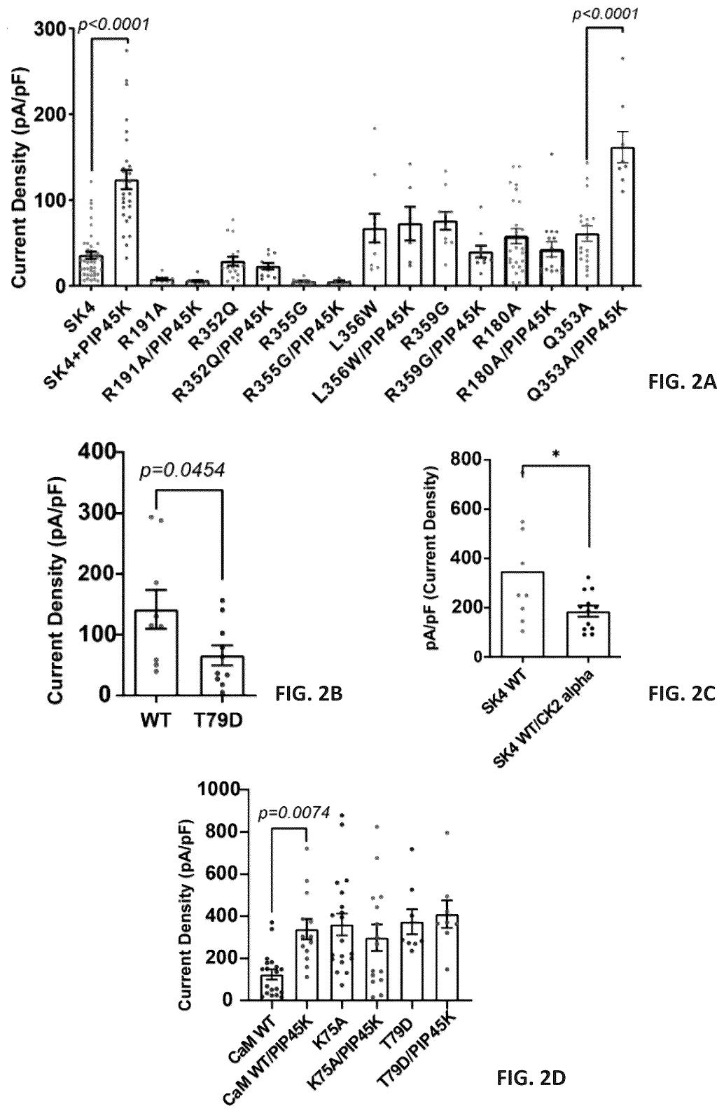

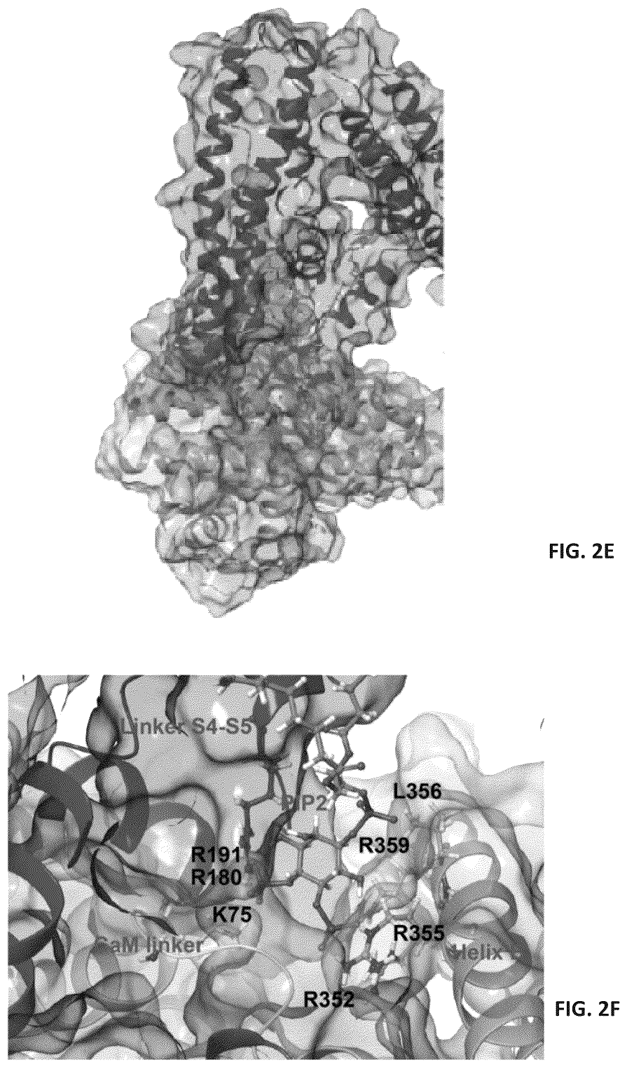

To further examine the role of PIP2 in SK4 channel activation, CHO cells were co-transfected with plasmid DNAs encoding for WT SK4 and for PIP4,5-kinase, that elevates PIP2 levels by producing PIP2 from phosphatidylinositol 4-phosphate (PI4P). The resulting K⁺ currents were recorded in the whole-cell configuration of the patch-clamp technique, in the presence of 1 μM internal free-Ca²⁺. The obtained data is presented in FIG. 2A (two leftmost bars) and show that in the presence of PIP4,5-kinase, the current density of WT SK4 channels was increased by 3.7-fold.

These data indicate that PIP2 is involved in SK4 channel gating and exhibits higher diC8-PIP2 affinity towards SK4 channel compared to its affinity towards SK2, which may result in high modulation sensitivity within a dynamic range of PIP2 and Ca²⁺ concentrations in physiological conditions.

It has been reported that in SK2 channels, phosphorylation of CaM at T79 by casein kinase II (CK2) reduces both the Ca²⁺ and the PIP2 sensitivity of the SK2/CaM channel complex for activation, thus suggesting that the levels of CaM phosphorylation could modulate the affinity of PIP2 and Ca²⁺ for the channel [see examples in: He et al., Front Cardiovasc Med (2021) 8; Wulff et al., Proc Natl Acad Sci USA (2000) 97, 8151-8156].

In order to probe whether phosphorylation of CaM at T79 also affects the activation of SK4 channels by Ca²⁺ and PIP2, the phosphomimetic mutant of CaM (T79D) was used. To rule out any effect of CaM on channel trafficking, purified recombinant proteins of WT CaM (3 μM) and CaM T79D (3 μM) were introduced into the patch pipette internal solution containing a saturating concentration of 5 μM free-Ca²⁺, and the K⁺ currents produced by WT SK4 channels were recorded. The obtained data is presented in FIG. 2B and show that a significantly lower current density (54% decrease) was obtained when the pipette solution contained recombinant CaM T79D (66 pA/pF), compared to WT CaM (142 pA/pF). As can be seen in FIG. 2C, co-expression of WT SK4 channels with the CK2 a subunit enzyme produced a 47% decrease in the current density with a pipette solution containing 5 μM free-Ca²⁺ and 5 mM ATP-K₂.

The ability of PIP2 to stimulate SK4 currents under conditions of CaM phosphorylation was tested. WT SK4 channels were co-expressed with either WT CaM or CaM T79D in the absence or presence of PIP4,5-kinase. As can be seen in FIG. 2D, in the presence of PIP4,5-kinase, the current density of WT SK4 channels significantly increased upon co-expression with WT CaM (2.6-fold) but not with CaM T79D (1.1-fold), as can be seen in FIG. 2D, two rightmost and two leftmost bar plots.

To conclude, these data suggest that PIP2 is involved in SK4 channel gating and exhibits higher diC8-PIP2 affinity towards SK4 channel compared to its affinity towards SK2. The data further suggest that similar to SK2 channels, phosphorylation of CaM at the T79 residue can lower the sensitivity of SK4 channel activation by PIP2 and Ca²⁺.

In order to examine the binding of PIP2 to the SK4 channel, molecular docking of PIP2 to the Ca²⁺-bound state I (6CNN) of the SK4 channel cryo-EM structure was simulated.

As shown in FIG. 2E, molecular docking of PIP2 to the Ca²⁺-bound state I (6CNN) of the SK4 channel cryo-EM structure indicated that the outward-facing fatty acid tail of PIP2 could fit into a gorge formed by the boundaries of S1, S2, S3, and S4 transmembrane helices, while the inward-facing phosphate head groups are in close proximity to the bottom of the PIP2 binding pocket formed by the S4-S5 linker, the CaM flexible linker and the helix B of the proximal C-terminus from the adjacent subunit. FIG. 2F illustrates the specific interactions between PIP2 and the residues in the SK4 binding pocket: In the S4-S5 linker, the guanidinium group of R180 residue interacts via H-bonding with the phosphate P4 of PIP2 (distance H—O=1.7 Å); R191 residue can also be engaged via its guanidinium functionality into H-bonding with the P4 of PIP2 (distance H—O=2.0 Å); In helix B of the neighboring subunit, the guanidinium groups of R352, R355, and R359 residues interact via H-bonding with the phosphate P5 of PIP2 (distances H—O=1.7 Å, 2 Å, and 2.5 Å, respectively); In the same region, a non-polar residue L356 is close to the PIP2 phosphodiester moiety (distance H—O=2.7 Å) and it is assumed that hydrophobic interactions may affect the positioning of the fatty acid tail in the gorge; and in the CaM interlobe linker, the F amine of the K75 residue interacts via H-bonding with the phosphate P4 of PIP2 (distance H—O=1.8 Å).

To examine ex-vivo the interactions between PIP2 and the simulated residues seen in FIG. 2F, the positively charged residues interacting with the oxygen of the PIP2 phosphate head groups were mutated, as well as other residues that were at atomic proximity of the PIP2 molecule such as L356 residue in helix B. The impact of the mutations on the current densities resulting from the co-expression of SK4 channels with PIP4,5-kinase were measured using whole-cell patch-clamp recording, with 1 μM free-Ca²⁺ in the pipette internal solution, as can be seen in FIG. 2A and FIG. 2C. The mutants R180 Å, R191A of linker S4-S5, and R352Q, R355G, R359G of helix B, which remove the positive charge, were unable to be activated by the PIP4,5-kinase (1.6-, 0.8-, 0.8-, 1-, and 0.5-fold, respectively) as compared to WT SK4 (3.7-fold), as presented in FIG. 2A. Mutant K75A of the CaM linker co-expressed with WT SK4 channel was also unable to be activated by the PIP4,5-kinase (0.7-fold), as can be seen in FIG. 2D, two middle bars. The current densities of mutants R191A and R355G were smaller than that of the WT both in the absence and presence of PIP4,5-kinase, indicating that these residues are crucial for PIP2 gating function, as can be seen in FIG. 2A. Mutant L356W of helix B is insensitive to activation by PIP4,5-kinase (1.1-fold), suggesting that this residue, in proximity to the PIP2 phosphodiester bond, plays a role in the docking of PIP2 into the gorge of the S1-S4 transmembrane region. The mutant of residue Q353 (Q353A), which does not interact with PIP2 and is the close neighbor of residue R352, is potently activated by PIP4,5-kinase (3.5-fold), as indicated by FIG. 2A, underscoring the specificity of PIP2 interaction within its binding pocket.

The functional validation of the PIP2 molecular docking establishes PIP2 as a gating molecule being engaged mainly via electrostatic interactions and binding to a pocket formed by the gorge within the S1-S4 helices, the S4-S5 linker, the CaM linker region, and the helix B of the proximal C-terminus.

### Example 2

### Design and Synthesis of SK4 Allosteric Modulators

Inhibiting SK4 channel was previously indicated to be a point of interest. In view of the suggested role of PIP2 in activating SK4 channels, the PIP2-binding domain of the channel can be considered to serve as a target for therapeutic small molecules.

In a search for small molecules that can target the PIP2-binding domain to the SK4 channel, the present inventors have sought of manipulating structures of known compounds that activate SK channels by binding in proximity to the PIP2-binding pocket, such as 1-EBIO, DCEBIO, Riluzol, NS309 and SKA-31, as presented below [Pedersen et al. Biochimica et Biophysica Acta (BBA)—Biomembranes (1999) 1420 (1-2), 231-240; Singh et al. Journal of Pharmacology and Experimental Therapeutics (2001), 296 (2) 600-611; Liu et al. Journal of Neuroimmune Pharmacology (2013) 8, 227-237; Chen et al. Front. Pharmacol. (2019) 10, 1432; and Sankaranarayanan et al. Molecular Pharmacology, (2009), 75 (2) 281-295].

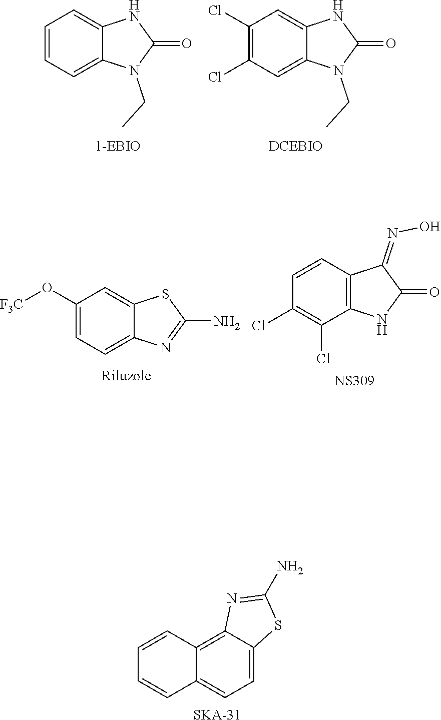

While all these compounds share several structural features, the present inventors have used benzimidazole and benzoxazole skeletons for the design and preparation of compounds and have studied the effect of structural manipulations of these base skeletons on SK4 channel.

The synthetic pathways for preparing such exemplary compounds are described in further detail hereinafter. Once prepared, the obtained compounds were purified using one of the following purification procedures:

### Purification Procedure 1:

Compounds were submitted to a reverse phase flash purification procedure using a Biotage® SNAP Ultra C18 cartridge. The Biotage® SNAP Ultra C18 60 g cartridge (HP-Sphere, 25 μm particle size) was mounted on a fully automated flash chromatography instrument (Biotage Isolera One). The system was equipped with an expanded fraction collector bed and dual wave length UV—V detector. For the purification, the crude powder was dissolved in the DMSO. Then, 1 mL of the resulted solution were loaded onto the cartridge. The elution process was done at a flow rate of 50 mL/minute and 20 mL of fraction were collected per tube by UV absorbance at 254 nm. All the chromatographic procedure was performed using a linear gradient solvent system. The elution started by equilibrating the column with 95% of water (solvent A) and 2 CV of 5% of acetonitrile (solvent B). Then, the cartridge was eluted with CV of the mobile phase starting from 5% to 100% of solvent B.

### Purification Procedure 2:

Compounds were submitted to a normal phase flash purification procedure using a silica Biotage® SNAP Ultra cartridge. The Biotage® SNAP Ultra C18 12 g cartridge (HP-Sphere, 25 μm particle size) was mounted on a fully automated flash chromatography instrument (Biotage Isolera One). The system was equipped with an expanded fraction collector bed and dual wave length UV-Vis detector. For the purification, the crude powder adsorbate into silica. Then, adsorbate crude transferred to empty Biotage® DVL column and equipment with DLV Plunger. The elution process was done at a flow rate of 36 mL/minute and 20 mL of fraction were collected per tube by UV absorbance at 254 nm. All the chromatographic procedure was performed using a linear gradient solvent system. The elution started by equilibrating the column with 95% of hexane (solvent A) and 2 CV of 5% of ethyl acetate (solvent B). Then, the cartridge was eluted with 10 CV of the mobile phase starting from 5% to 100% of solvent B.

### Purification Procedure 3:

Compounds were submitted to a reverse phase HPLC purification procedure using a Waters AutoPurification system. The preparative module equipped with SQD2 MS detector at the following conditions: Waters XSelect CSH130 C18 5 m 19×250 mm OBD column using a 20 minutes gradient from 95:5 water:acetonitrile (both with 0.1% formic acid) to 100% acetonitrile (0.1% formic acid).

The chemical structure of all the synthesized compounds was verified by ¹H-NMR and ¹³C-NMR.

### Synthesis of Benzimidazolone Compounds:

The general synthesis of benzimidazolone B, is depicted in Scheme 1 below:

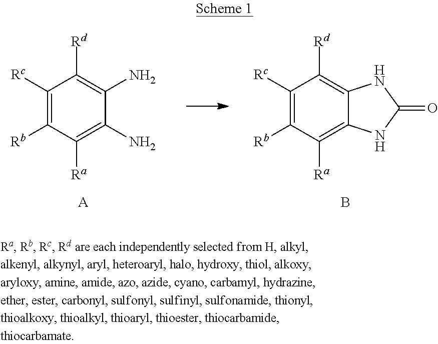

Phenyldiamine derivative A is reacted in the presence of a formylating agent (e.g., an amide-forming coupling agent such as CDI) in a polar solvent (e.g., a polar aprotic solvent such as, but not limited to, THF) to generate a benzimidazolone derivative B, using one of the following synthetic procedures denoted as general procedure 1.1 and 1.2.

Exemplary compounds that are synthesizable according to this general procedure include benzimidazolone derivatives B as depicted in Scheme 1 above, in which Ra is hydrogen, Rb is hydrogen, methyl or halogen, R^{c} is hydrogen, methyl, methoxy, nitrile, nitro, chloride, bromide or fluoride, and R^{d} is hydrogen, methyl or nitro.

Compound synthesized according to this synthetic pathways include compounds BA10, BA20, BA30, BA40, BA50, BA6-, BA70, BA80, BA90, BA100, as specified in Table 1.

General procedure 1.1: In a reaction vessel equipped with magnetic stirrer and an addition funnel, phenyldiamine derivative A is dissolved in a polar aprotic solvent (e.g., tetrahydrofuran, THF). An amide-forming coupling agent (e.g., CDI) is added to the obtained solution dropwise through the addition funnel while cooling (e.g. at 0° C.), and the resulting mixture is stirred at room temperature for several hours (e.g., overnight). The reaction progress is monitored by LC-MS. After completion of the reaction, the solid is filtered and dried under reduced pressure. Purification by chromatography yields the benzimidazolone derivative B.

General procedure 1.2: To a reaction vial equipped with a stirring bar, diamine derivative A, an amide-forming coupling agent (e.g., CDI), and a polar aprotic solvent (e.g., THF) is added, the vial is sealed and the reaction mixture is microwave-irradiated (ramp=2 minutes, Pmax=150 W) for several minutes (e.g., 20 minutes) at elevated temperature (e.g., at 180° C.). The reaction is monitored by LC-MS. After completion of the reaction, the solid is filtered and dried under reduced pressure. Purification by chromatography yields the benzimidazolone derivative B.

Table 1 below presents exemplary compounds which were prepared and characterized based on the general synthetic procedures 1.1 and 1.2.

TABLE 1

| | Synthetic | Purification | Yield | HPLC |
|---|---|---|---|---|
| Compound | method | procedure | (%) | purity (%) |
| 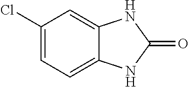 [SMILES: O=c1[nH]c2ccc(Cl)cc2[nH]1] | 1.2 | 1 | 83 | >95 |
| 5-chloro-1,3-dihydro-2H- | | | | |
| benzo[d]imidazol-2-one | | | | |
| BA10 | | | | |
| 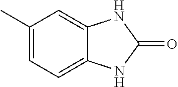 | 1.2 | 1 | 79 | >95 |
| 5-methyl-1,3-dihydro-2H- | | | | |
| benzo[d]imidazol-2-one | | | | |
| BA20 | | | | |
| 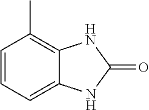 | 1.2. | 1 | 84 | >95 |
| 4-methyl-1,3-dihydro-2H- | | | | |
| benzo[d]imidazol-2-one | | | | |
| BA30 | | | | |
| 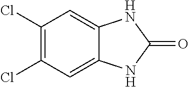 [SMILES: O=c1[nH]c2cc(Cl)c(Cl)cc2[nH]1] | 1.1. | 1 | 42 | >95 |
| 5,6-dichloro-1,3-dihydro-2H- | | | | |
| benzo[d]imidazol-2-one | | | | |
| BA40 | | | | |
| 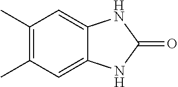 | 1.1. | 1 | 49.3 | >95 |
| 5,6-dimethyl-1,3-dihydro-2H- | | | | |
| benzo[d]imidazol-2-one | | | | |
| BA50 | | | | |
| 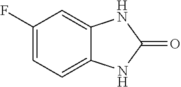 [SMILES: O=c1[nH]c2ccc(F)cc2[nH]1] | 1.2. | 1 | 57 | >95 |
| 5-fluoro-1,3-dihydro-2H- | | | | |
| benzo[d]imidazol-2-one | | | | |
| BA60 | | | | |
| 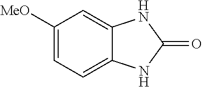 [SMILES: COc1ccc2[nH]c(=O)[nH]c2c1] | 1.2. | 1 | 31 | >95 |
| 5-methoxy-1,3-dihydro-2H- | | | | |
| benzo[d]imidazol-2-one | | | | |
| BA70 | | | | |
| 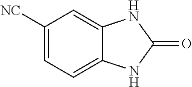 [SMILES: N#Cc1ccc2[nH]c(=O)[nH]c2c1] | 1.2. | 1 | 37 | >95 |
| 2-oxo-2,3-dihydro-1H- | | | | |
| benzo[d]imidazole-5-carbonitrile | | | | |
| BA80 | | | | |
| 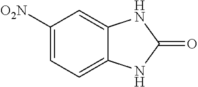 [SMILES: O=c1[nH]c2ccc([N+](=O)[O-])cc2[nH]1] | 1.2. | 1 | 54 | >95 |
| 5-nitro-1,3-dihydro-2H- | | | | |
| benzo[d]imidazol-2-one | | | | |
| BA90 | | | | |
| 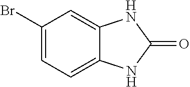 [SMILES: O=c1[nH]c2ccc(Br)cc2[nH]1] | 1.2. | 1 | 46 | >95 |
| 5-bromo-1,3-dihydro-2H- | | | | |
| benzo[d]imidazol-2-one | | | | |
| BA100 | | | | |

Following are exemplary representative synthetic protocols.

### Preparation of 5-bromo-1,3-dihydro-2H-benzo[d]imidazol-2-one (BA40)

In a reaction vessel equipped with a magnetic stirrer and addition funnel, 4,5-dichlorobenzene-1,2-diamine (1 mol equivalent, 3 mmol, 531 mg) was dissolved in anhydrous tetrahydrofuran (THF) (20 ml). CDI (1.5 mol equivalent, 4.5 mmol, 730 mg, 0.45 M in THF) was added dropwise through the addition funnel at 0° C., and the resulting mixture was stirred at room temperature overnight. The reaction was monitored by LC-MS. After completion of the reaction, the solid was filtered, dried under vacuum, and purified by purification procedure 1, yielding BA40 as a white solid.

¹H NMR (500 MHz, DMSO-d₆) δ 10.92 (s, 2H), 7.12 (s, 2H).

¹³C NMR (126 MHz, DMSO-d₆) δ 155.12, 129.78, 122.29, 109.67.

### Preparation of 5-bromo-1,3-dihydro-2H-benzo[d]imidazol-2-one (BA100)

Into a 10 ml process vessel equipped with a stirring bar, 4-bromobenzene-1,2-diamine (1 mol equivalent, 3 mmol, 561 mg), CDI (6 equiv., 6 mmol, 973 mg) and THF (4 ml) were added. The vial was sealed and the reaction mixture was microwave-irradiated for 20 minutes at 180° C. The reaction was monitored by LC-MS. After completion of the reaction, the solvent was evaporated under reduced pressure, and the residue was neutralized with aqueous HCl (10 ml, 2N), the solution was decanted, washed again with water (10 ml) and dried under vacuum. Purification was performed by purification procedure 1, yielding BA100 as a white solid.

¹H NMR (500 MHz, DMSO-d₆) δ 10.76 (s, 2H), 7.08 (dd, J=8.2, 1.0 Hz, 1H), 7.05 (d, J=1.0 Hz, 1H), 6.87 (d, J=8.3 Hz, 1H).

¹³C NMR (126 MHz, DMSO-d₆) δ 154.91, 131.09, 128.87, 122.77, 111.86, 110.90, 109.94.

### Synthesis of Substituted Benzimidazolone:

The general synthesis of mono- and di-N-alkyl benzimidazolone is depicted in Scheme 2 below:

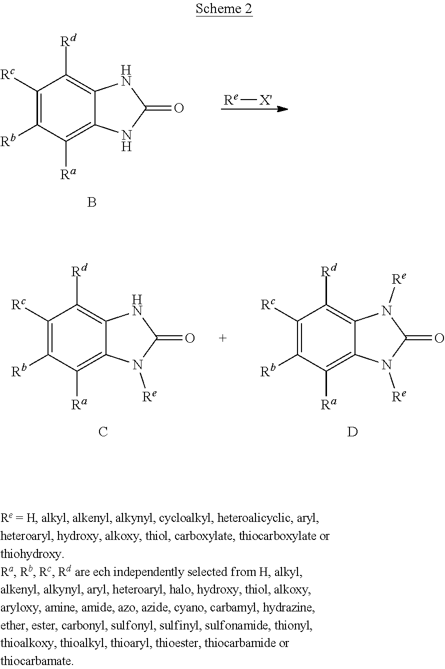

Generally, a benzimidazolone derivatives B is reacted in the presence of an alkylating agent to generate a mono- and/or di-N-alkyl benzimidazolone derivative C and/or D, using one of the following synthetic procedures denoted as procedure 2.1 and 2.2.

Exemplary compounds that are synthesizable according to this general procedure include mono-alkylated benzimidazolone derivatives C and bis-alkylated benzimidazolone derivatives D as depicted in Scheme 2 above, in which R^{c} is hydrogen or alkyl, Ra and R^{d} are each independently hydrogen or nitro, R^{c} is hydrogen, methyl or chloride, and R^{d} is hydrogen, methyl, chloride or fluoride.

Compounds synthesized according to this synthetic pathways include compounds BA2a, BA3a, BA3b, BA4a, BA4b, BA5a, BA5b, BA6a, BA6b, BA7a, BA7b, BA8a, BA8b, BA9a, BA9b, BA20C1c, BA20C3c, BA23, BA26, BA29, BA41, BA42, BA43, BA44, BA45, BA46, BA53, BA54, BA55, BA56, BA63, BA66, BA69, as specified in Table 2.

General procedure 2.1: To a solution of benzimidazole B in a polar aprotic solvent (e.g., dimethylformamide, DMF), a base (e.g., potassium carbonate) is added. The reaction mixture is cooled (e.g., to 0° C.), an alkylating agent (e.g., an alkyl halide) is thereafter added slowly, and the obtained solution is stirred at room temperature for several hours (e.g., overnight). Upon reaction completion, water is added, the resulting suspension is filtered, and the collected solids are washed with water and then dried under reduced pressure to obtain a mono-N-alkylated benzimidazolone derivative C and/or di-N-alkylated benzimidazolone derivative D.

General procedure 2.2: To a solution of a benzimidazole B in a polar aprotic solvent (e.g., DMF), a base (e.g., potassium carbonate) is added. The reaction mixture is cooled (e.g., to 0° C.), an alkylating agent (e.g., alkyl halide) is thereafter slowly added and obtained the solution is stirred at room temperature for several hours (e.g., overnight). Upon reaction completion, water is added, the solvents are evaporated and the crude is dissolved in a polar solvent (e.g., dimethylsulfoxide, DMSO), centrifuged, and the solvent is decanted. The precipitated mono-N-alkylated benzimidazolone derivative C and/or di-N-alkylated benzimidazolone derivative D is/are dried under vacuum.

Table 2 below presents exemplary compounds which were prepared and characterized based on the general synthetic procedures 2.1 and 2.2.

TABLE 2

| | Synthetic | Purification | Yield | HPLC |
|---|---|---|---|---|
| Compound | method | procedure | (%) | purity (%) |
| 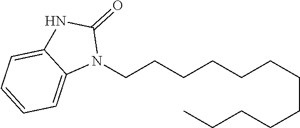 [SMILES: CCCCCCCCCCCCn1c(=O)[nH]c2ccccc21] | 2.2. | 3 | 0.5 | >95 |
| 1-dodecyl-1,3-dihydro-2H- | | | | |
| benzo[d]imidazol-2-one | | | | |
| BA9B | | | | |
| 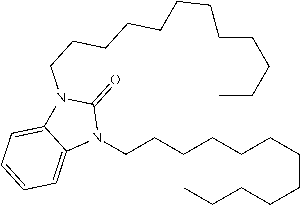 [SMILES: CCCCCCCCCCCCn1c(=O)n(CCCCCCCCCCCC)c2ccccc21] | 2.2. | 3 | 2.5 | >95 |
| 1,3-didodecyl-1,3-dihydro- | | | | |
| 2H-benzo[d]imidazol-2-one | | | | |
| BA9A | | | | |
| 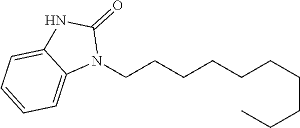 [SMILES: CCCCCCCCCCn1c(=O)[nH]c2ccccc21] | 2.2. | 3 | 0.5 | >95 |
| 1-decyl-1,3-dihydro-2H- | | | | |
| benzo[d]imidazol-2-one | | | | |
| BA8B | | | | |
| 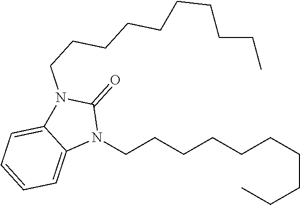 [SMILES: CCCCCCCCCCn1c(=O)n(CCCCCCCCCC)c2ccccc21] | 2.2. | 3 | 4 | >95 |
| 1,3-didecyl-1,3-dihydro-2H- | | | | |
| benzo[d]imidazol-2-one | | | | |
| BA8A | | | | |
| 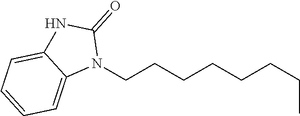 [SMILES: CCCCCCCCn1c(=O)[nH]c2ccccc21] | 2.2. | 3 | 1 | >95 |
| 1-octyl-1,3-dihydro-2H- | | | | |
| benzo[d]imidazol-2-one | | | | |
| BA7B | | | | |
| 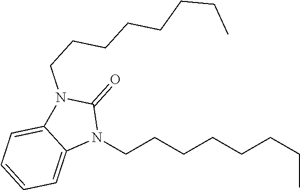 [SMILES: CCCCCCCCn1c(=O)n(CCCCCCCC)c2ccccc21] | 2.2. | 3 | 3 | >95 |
| 1,3-dioctyl-1,3-dihydro-2H- | | | | |
| benzo[d]imidazol-2-one | | | | |
| BA7A | | | | |
| 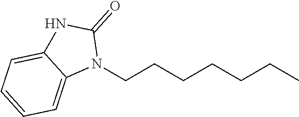 [SMILES: CCCCCCCn1c(=O)[nH]c2ccccc21] | 2.2. | 3 | 1 | >95 |
| 1-heptyl-1,3-dihydro-2H- | | | | |
| benzo[d]imidazol-2-one | | | | |
| BA6B | | | | |
| 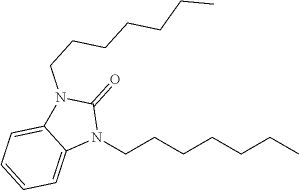 [SMILES: CCCCCCCn1c(=O)n(CCCCCCC)c2ccccc21] | 2.2. | 3 | 3 | >95 |
| 1,3-diheptyl-1,3-dihydro-2H- | | | | |
| benzo[d]imidazol-2-one | | | | |
| BA6A | | | | |
| 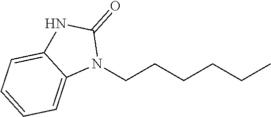 [SMILES: CCCCCCn1c(=O)[nH]c2ccccc21] | 2.2. | 3 | 1 | >95 |
| 1-hexyl-1,3-dihydro-2H- | | | | |
| benzo[d]imidazol-2-one | | | | |
| BA5B | | | | |
| 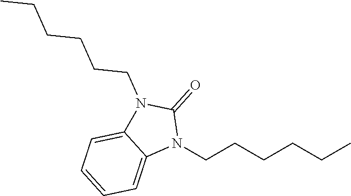 [SMILES: CCCCCCn1c(=O)n(CCCCCC)c2ccccc21] | 2.2. | 3 | 2 | >95 |
| 1,3-dihexyl-1,3-dihydro-2H- | | | | |
| benzo[d]imidazol-2-one | | | | |
| BA5A | | | | |
| 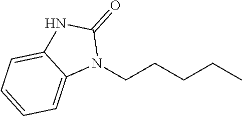 | 2.1. | 1 | 7 | >95 |
| 1-pentyl-1,3-dihydro-2H- | | | | |
| benzo[d]imidazol-2-one | | | | |
| BA4B | | | | |
| 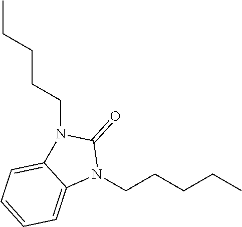 [SMILES: CCCCCn1c(=O)n(CCCCC)c2ccccc21] | 2.1. | 1 | 5 | >95 |
| 1,3-dipentyl-1,3-dihydro-2H- | | | | |
| benzo[d]imidazol-2-one | | | | |
| BA4A | | | | |
| 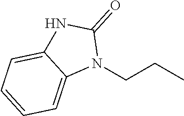 | 2.2. | 1 | 4 | >95 |
| 1-propyl-1,3-dihydro-2H- | | | | |
| benzo[d]imidazol-2-one | | | | |
| BA3B | | | | |
| 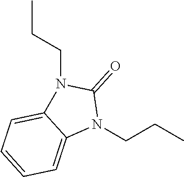 [SMILES: CCCn1c(=O)n(CCC)c2ccccc21] | 2.2. | 1 | 19 | >95 |
| 1,3-dipropyl-1,3-dihydro-2H- | | | | |
| benzo[d]imidazol-2-one | | | | |
| BA3A | | | | |
| 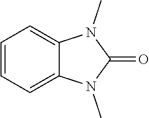 | 2.2. | 1 | 19 | >95 |
| 1,3-dimethyl-1,3-dihydro-2H- | | | | |
| benzo[d]imidazol-2-one | | | | |
| BA2A | | | | |
| 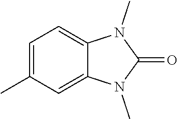 | 2.2. | 1 | 28 | >95 |
| 1,3,5-trimethyl-1,3-dihydro- | | | | |
| 2H-benzo[d]imidazol-2-one | | | | |
| BA2OC1c | | | | |
| 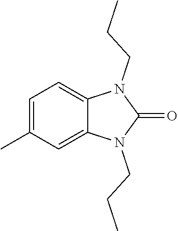 [SMILES: CCCn1c(=O)n(CCC)c2cc(C)ccc21] | 2.2. | 1 | 15 | >95 |
| 5-methyl-1,3-dipropyl-1,3- | | | | |
| dihydro-2H- | | | | |
| benzo[d]imidazol-2-one | | | | |
| BA2OC3c | | | | |
| 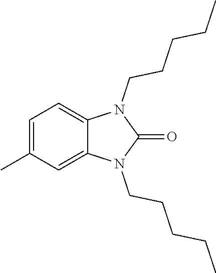 [SMILES: CCCCCn1c(=O)n(CCCCC)c2cc(C)ccc21] | 2.2. | 1 | 31 | >95 |
| 5-methyl-1,3-dipentyl-1,3- | | | | |
| dihydro-2H- | | | | |
| benzo[d]imidazol-2-one | | | | |
| BA23 | | | | |
| 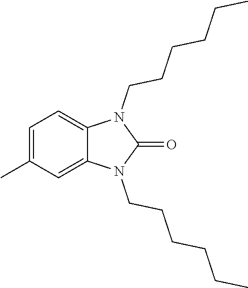 [SMILES: CCCCCCn1c(=O)n(CCCCCC)c2cc(C)ccc21] | 2.2. | 1 | 27 | >95 |
| 1,3-dihexyl-5-methyl-1,3- | | | | |
| dihydro-2H- | | | | |
| benzo[d]imidazol-2-one | | | | |
| BA26 | | | | |
| 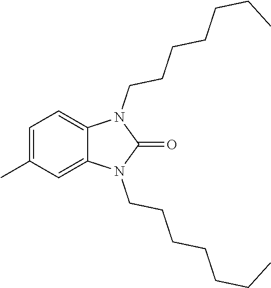 [SMILES: CCCCCCCn1c(=O)n(CCCCCCC)c2cc(C)ccc21] | | | | |
| 1,3-diheptyl-5-methyl-1,3- | 2.2. | 1 | 15 | >95 |
| dihydro-2H- | | | | |
| benzo[d]imidazol-2-one | | | | |
| BA29 | | | | |
| 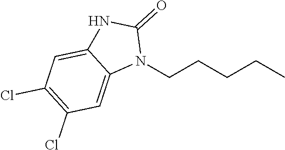 [SMILES: CCCCCn1c(=O)[nH]c2cc(Cl)c(Cl)cc21] | 2.2. | 3 | 9 | >95 |
| 5,6-dichloro-1-pentyl-1,3-dihydro-2H- | | | | |
| benzo[d]imidazol-2-one | | | | |
| BA41 | | | | |
| 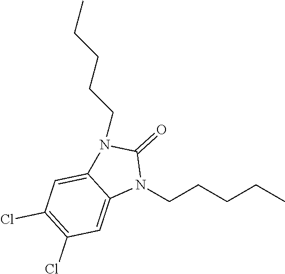 [SMILES: CCCCCn1c(=O)n(CCCCC)c2cc(Cl)c(Cl)cc21] | 2.2. | 3 | 41 | >95 |
| 5,6-dichloro-1,3-dipentyl-1,3-dihydro- | | | | |
| 2H-benzo[d]imidazol-2-one | | | | |
| BA42 | | | | |
| 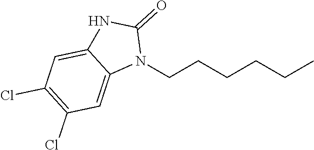 [SMILES: CCCCCCn1c(=O)[nH]c2cc(Cl)c(Cl)cc21] | 2.2. | 3 | 15 | >95 |
| 5,6-dichloro-1-hexyl-1,3-dihydro-2H- | | | | |
| benzo[d]imidazol-2-one | | | | |
| BA43 | | | | |
| 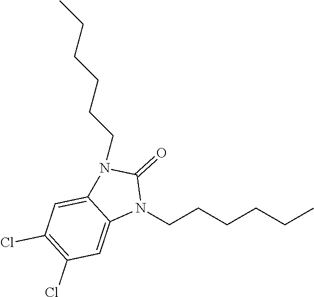 [SMILES: CCCCCCn1c(=O)n(CCCCCC)c2cc(Cl)c(Cl)cc21] | 2.2. | 3 | 7 | >95 |
| 5,6-dichloro-1,3-dihexyl-1,3-dihydro- | | | | |
| 2H-benzo[d]imidazol-2-one | | | | |
| BA44 | | | | |
| 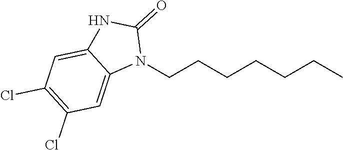 [SMILES: CCCCCCCn1c(=O)[nH]c2cc(Cl)c(Cl)cc21] | 2.2. | 3 | 7 | >95 |
| 5,6-dichloro-1-heptyl-1,3-dihydro-2H- | | | | |
| benzo[d]imidazol-2-one | | | | |
| BA45 | | | | |
| 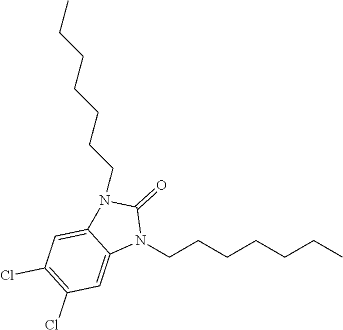 [SMILES: CCCCCCCn1c(=O)n(CCCCCCC)c2cc(Cl)c(Cl)cc21] | 2.2. | 3 | 12 | >95 |
| 5,6-dichloro-1,3-diheptyl-1,3-dihydro- | | | | |
| 2H-benzo[d]imidazol-2-one | | | | |
| BA46 | | | | |
| 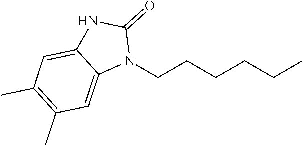 [SMILES: CCCCCCn1c(=O)[nH]c2cc(C)c(C)cc21] | 2.2. | 3 | 17 | >94 |
| 1-hexyl-5,6-dimethyl-1,3-dihydro-2H- | | | | |
| benzo[d]imidazol-2-one | | | | |
| BA53 | | | | |
| 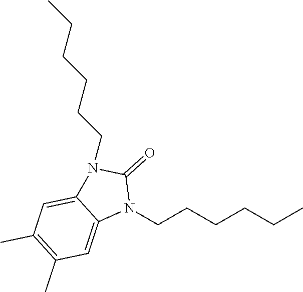 [SMILES: CCCCCCn1c(=O)n(CCCCCC)c2cc(C)c(C)cc21] | 2.2. | 3 | 5 | >95 |
| 1,3-dihexyl-5,6-dimethyl-1,3-dihydro- | | | | |
| 2H-benzo[d]imidazol-2-one | | | | |
| BA54 | | | | |
| 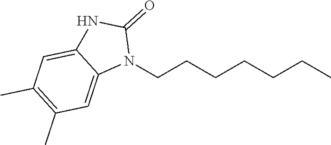 [SMILES: CCCCCCCn1c(=O)[nH]c2cc(C)c(C)cc21] | 2.2. | 3 | 8 | >95 |
| 1-heptyl-5,6-dimethyl-1,3-dihydro-2H- | | | | |
| benzo[d]imidazol-2-one | | | | |
| BA55 | | | | |
| 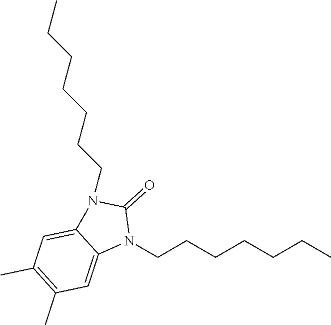 [SMILES: CCCCCCCn1c(=O)n(CCCCCCC)c2cc(C)c(C)cc21] | 2.2. | 3 | 4 | >95 |
| 1,3-diheptyl-5,6-dimethyl-1,3-dihydro- | | | | |
| 2H-benzo[d]imidazol-2-one | | | | |
| BA56 | | | | |
| 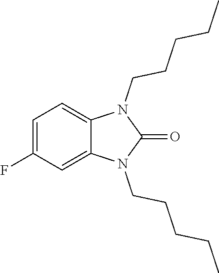 [SMILES: CCCCCn1c(=O)n(CCCCC)c2cc(F)ccc21] | 2.2. | 3 | 43 | >95 |
| 5-fluoro-1,3-dipentyl-1,3-dihydro-2H- | | | | |
| benzo[d]imidazol-2-one | | | | |
| BA63 | | | | |
| 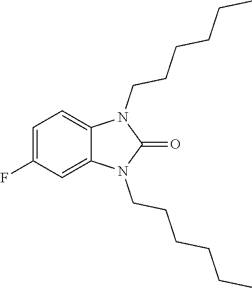 [SMILES: CCCCCCn1c(=O)n(CCCCCC)c2cc(F)ccc21] | 2.2. | 3 | 35 | >95 |
| 5-fluoro-1,3-dihexyl-1,3-dihydro-2H- | | | | |
| benzo[d]imidazol-2-one | | | | |
| BA66 | | | | |
| 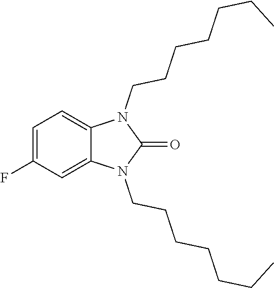 [SMILES: CCCCCCCn1c(=O)n(CCCCCCC)c2cc(F)ccc21] | 2.2. | 3 | 36 | >95 |
| 5-fluoro-1,3-diheptyl-1,3-dihydro-2H- | | | | |
| benzo[d]imidazol-2-one | | | | |
| BA69 | | | | |

Following are exemplary representative synthetic protocols.

### Preparation of 1-octyl-1,3-dihydro-2H-benzo[d]imidazol-2-one and 1,3-dioctyl-1,3-dihydro-2H-benzo[d]imidazol-2-one (BA7B) and 1,3-dioctyl-1,3-dihydro-2H-benzo[d]imidazol-2-one (BA7A)

To a solution of commercial 1,3-dihydro-2H-benzo[d]imidazol-2-one (1 mol equivalent, 1.18 mmol, 128 mg) in dimethylformamide (DMF) (3 ml), potassium carbonate (2 mol equivalent, 2.37 mmol, 464 mg) was added. The reaction mixture was cooled to 0° C., then 1-iodododecane (2 mol equivalent, 2.37 mmol, 430 μl) was slowly added, and the solution was stirred at room temperature overnight. Upon reaction completion, water was added. The solvents evaporated and the crude dissolved in dimethylsulfoxide (1 ml). The precipitated solid was centrifuged and the solvent was decanted. The resulting solid was dried under vacuum, and purified by purification procedure 3, yielding BA7B as a white solid, and BA7A as a white solid. The compound's structure was verified by NMR.

### Preparation of 1-heptyl-1,3-dihydro-2H-benzo[d]imidazol-2-one and 1,3-diheptyl-1,3-dihydro-2H-benzo[d]imidazol-2-one (BA6B) and 1,3-diheptyl-1,3-dihydro-2H-benzo[d]imidazol-2-one (BA6A)

To a solution of commercial 1,3-dihydro-2H-benzo[d]imidazol-2-one (1 mol equivalent, 2.8 mmol, 303 mg) in dimethylformamide (3 ml), potassium carbonate (1.2 mol equivalent, 3.3 mmol, 464 mg) was added. The reaction mixture was cooled to 0° C., then 1-iodoheptane (1.2 mol equivalent, 2.3 mmol, 551 μl) was slowly added, and the solution was stirred at room temperature overnight. Upon reaction completion, water was added. The solvents evaporated and the crude dissolved in dimethylsulfoxide (1 ml). The precipitated solid was centrifuged and the solvent was decanted. The resulting solid was dried under vacuum, and purified by purification procedure 3, yielding BA6B as a white solid, and BA6A as a white.

¹H NMR (400 MHz, DMSO-d₆) δ 10.78 (s, 1H), 7.13-7.06 (m, 1H), 7.02-6.93 (m, 3H), 3.76 (t, J=7.1 Hz, 2H), 1.68-1.55 (m, 2H), 1.33-1.15 (m, 9H), 0.84 (t, J=6.9 Hz, 3H).

¹³C NMR (101 MHz, DMSO-d₆) δ 154.62, 130.67, 128.64, 121.03, 120.85, 120.78, 109.06, 108.86, 108.06, 31.59, 28.70, 28.21, 26.53, 22.40, 14.31.

### Preparation of 1-hexyl-1,3-dihydro-2H-benzo[d]imidazol-2-one and 1,3-dihexyl-1,3-dihydro-2H-benzo[d]imidazol-2-one (BA5B) and 1,3-dihexyl-1,3-dihydro-2H-benzo[d]imidazol-2-one (BA5A)

To a solution of 1,3-dihydro-2H-benzo[d]imidazol-2-one (1 mol equivalent, 1.18 mmol, 128 mg) in dimethylformamide (10 ml), potassium carbonate (2 mol equivalent, 2.3 mmol, 330 mg) was added. The reaction mixture was cooled to 0° C., then 1-iodohexane (2 mol equivalent, 2.37 mmol, 349 μl) was slowly added, and the solution was stirred at room temperature overnight. Upon reaction completion, water was added. The solvents evaporated and the crude dissolved in dimethylsulfoxide (1 ml). The precipitated solid was centrifuged and the solvent was decanted. The resulting solid was dried under vacuum, and purified by purification procedure 3, yielding BA5B as a white solid, and BA5A as a white solid. The compound's structure was verified by NMR.

### Preparation of 1-pentyl-1,3-dihydro-2H-benzo[d]imidazol-2-one and 1,3-dipentyl-1,3-dihydro-2H-benzo[d]imidazol-2-one (BA4B) and 1,3-dipentyl-1,3-dihydro-2H-benzo[d]imidazol-2-one (BA4A)

BA4B was prepared according to General Procedure 2. To a solution of 1,3-dihydro-2H-benzo[d]imidazol-2-one (1 mol equivalent, 0.55 mmol, 60 mg) in dimethylformamide (10 ml), potassium carbonate (2 mol equivalent, 1.1 mmol, 150 mg) was added. The reaction mixture was cooled to 0° C., then 1-iodopentane (3 mol equivalent, 1.65 mmol, 217 μL) was slowly added, and the solution was stirred at room temperature overnight. Upon reaction completion, water was added. The resulting suspension was filtered and the collected solid washed with water (20 mL) and dried under vacuum. Purified by purification procedure 1, yielding BA4B as a white solid, and BA4A as a white solid.

¹H NMR (400 MHz, DMSO-d₆) δ 10.79 (s, 1H), 7.15-7.02 (m, 1H), 7.02-6.91 (m, 3H), 3.75 (t, J=7.1 Hz, 2H), 1.70-1.53 (m, 2H), 1.26 (ddt, J=18.3, 8.7, 5.4 Hz, 5H), 0.83 (t, J=7.0 Hz, 3H).

¹³C NMR (101 MHz, DMSO-d₆) δ 154.11, 130.14, 128.12, 120.54, 120.36, 108.56, 107.56, 30.60, 28.25, 27.42, 21.68, 13.78.

### Synthesis of N-Substituted Benzothiazolone, Benzimidazolone, and Benzoxazolone Derivatives

The general synthesis of N-substituted benzimidazolones and benzoxazolones using microwave irradiation is depicted in Scheme 3 below:

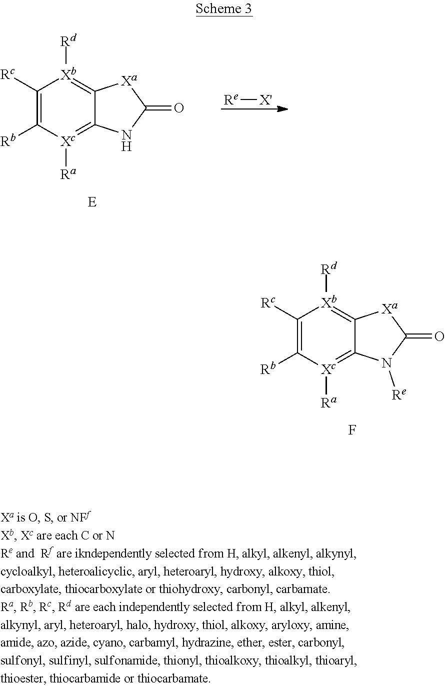

Generally, benzothiazolone, benzimidazolone, or benzoxazolone derivative E is reacted in the presence of an alkylating agent to generate a substituted benzothiazolone, benzimidazolone or benzoxazolone derivative F.

Exemplary compounds that are synthesizable according to this general procedure include benzoxazolone derivatives E as depicted in Scheme 3 above, in which X^{a} is oxygen, nitrogen or a protected nitrogen, X^{b} and X^{e} are each independently carbon or nitrogen, R^{e} is alkyl or CHC(O)NHCH₄H₈, R^{a} is a hydrogen or nitro, R^{b} and R^{c} are each hydrogen, and R^{d} is hydrogen or nitro.

More specifically, a benzimidazole, benzothiazole or benzoxazole E, carbonate base and an alkylating agent R^{e}—X′ as defined herein are placed in a 10 ml process vial, equipped with a stirring bar. A polar aprotic solvent (e.g., DMF) is added and the obtained solution is stirred at room temperature. Then, the vial is fitted with a Snap-On cap, and the mixture is heated (e.g., at 60° C.) under microwave irradiation (ramp=2 minutes, Pmax=150 watt) for several hours at elevated temperature (e.g., at 60° C.). The resulting mixture is partitioned between an organic solvent (e.g., dichloromethane (DCM)) and water. The aqueous layer is extracted with the organic solvent, and the combined organic layers are washed with water and brine, dried over Na₂SO₄, filtered, and concentrated under reduced pressure. Purification by chromatography methods yields the corresponding N-alkylated benzoxazolone F.

Based on the above-described general synthetic pathway, the following exemplary compounds were prepared.

### Preparation of 3-heptylbenzo[d]oxazol-2(3H)-one (BA6B9)

Benzo[d]oxazol-2(3H)-one (1 mol equivalent, 0.74 mmol, 100 mg), potassium carbonate (3 mol equivalent, 2.2 mmol, 307 mg), and 1-iodoheptane (5 mol equivalent, 3.7 mmol, 607 μl) were placed in a 10 ml process vial, equipped with a stirring bar. Dimethylformamide (2 ml) was added and the obtained solution was stirred for 30 seconds at room temperature. Then, the vial was fitted with a Snap-On cap, and the mixture was heated under microwave irradiation (ramp=2 minutes, Pₘₐₓ=150 W) for 3 hours at 60° C. The resulting mixture was partitioned between dichloromethane (DCM) and water, the aqueous layer was extracted three times with DCM, and the combined organic layers were washed with water and brine, dried over Na₂SO₄, filtered, and concentrated under reduced pressure. Purification by purification procedure 2 yielded BA6B9 as a white solid.

¹H NMR (400 MHz, DMSO-d₆) δ 7.32 (ddd, J=12.0, 7.8, 1.2 Hz, 2H), 7.22 (td, J=7.7, 1.2 Hz, 1H), 7.13 (td, J=7.8, 1.4 Hz, 1H), 3.81 (t, J=7.1 Hz, 2H), 1.68 (p, J=7.1 Hz, 2H), 1.32-1.20 (m, 8H), 0.83 (t, J=6.8 Hz, 3H).

¹³C NMR (101 MHz, DMSO-d₆) δ 142.36, 131.46, 124.31, 122.55, 110.04, 109.60, 42.05, 31.52, 28.59, 27.50, 26.33, 22.38, 14.29.

Some of the compounds were tested by comparing trace of WT SK4 channel currents in the absence and presence of 20 μM of the tested compound.

The modulations by each of the exemplary compounds are summarized in Table 3 below.

TABLE 3

| Compound | SK4 K⁺ channel modulating activity (IC₅₀) |
|---|---|
| BA10 | N/A |
| BA20 | Inactive |
| BA30 | Inactive |
| BA40 | Activator |
| BA50 | Inactive |
| BA100 | Activator |
| BA9B | N/A |
| BA9A | Inactive |
| BA8B | Inactive |
| BA8A | Inactive |
| BA7B | Inactive |
| BA7A | Inactive |
| BA6B | Inhibitor (50 μM) |
| BA6A | Inactive |
| BA5B | Inhibitor |
| BA5A | Inactive |
| BA4B | Inhibitor |
| BA4A | Inactive |
| BA3B | Inactive |
| BA3A | Inactive |
| BA2A | Inactive |
| BA2OC1c | Inactive |
| BA2OC3c | Inactive |
| BA23 | Inactive |
| BA26 | Inactive |
| BA29 | Inactive |
| BA41 | N/A |
| BA42 | Inactive |
| BA43 | N/A |
| BA44 | Inactive |
| BA45 | N/A |
| BA46 | Inactive |
| BA53 | N/A |
| BA54 | Inactive |
| BA55 | N/A |
| BA56 | Inactive |
| BA63 | Inactive |
| BA66 | Inactive |
| BA69 | Inactive |
| BA6b9 | Inhibitor (8.6 μM) |

As can be seen, benzimidazolone derivatives B (see, Scheme 1), phenyl-substituted with either alkyls, halogens or heteroatom-based groups, were mostly inactive, with the exception of BA40 and BA100, which showed activation of SK4 K⁺ channels. Benzimidazolone derivatives D (see, Scheme 2), bearing two linear N-alkyl chains, were generally inactive on SK4 channels. Among benzimidazolone derivatives C (see, Scheme 2), bearing a single linear N-alkyl chain, SK4 channel inhibition was demonstrated by analogs comprising C5-C7 alkyl chains, such as the exemplary compounds BA4B, BA5B, and BA6B (see, Table 2). Exemplary data is presented in FIG. 3A and in FIG. 3B. These data indicate that an exemplary benzimidazolone derivative B, BA40, activates SK4 channel by about 1.4-fold at 10 μM, while an exemplary benzoxazolone derivative C, BA6B, inhibited SK4 currents by 25% at 20 μM.

Some of the exemplary compounds, BA40, BA100, and BA6B, were compared by means of statistical summary of the pharmacological effects thereof on WT SK4 K⁺ currents, and the obtained data is presented in FIG. 3C. As can be seen, 10 μM BA40 led to 42% increase in current, 10 μM BA100 increased the current by 28%, and 20 μM BA6B provided an opposite trend as current decreased by 25%.

The dose-dependent inhibition of WT SK4 channels by the exemplary compound BA6b9 was tested. FIG. 4A (see, ‘BA6b9’ labeled plot, black dots) presents normalized SK4 currents following exposure to the exemplary compounds BA6b9 (10 μM) as a function of Ca²⁺ concentrations, lowers the channel's sensitivity to Ca²⁺ with an EC₅₀ for Ca²⁺ of 435 nM. The data presented in FIG. 4B also show that BA6b9 exhibits blocking activity with an IC₅₀ of 8.6 μM.

Further testing of BA6b9 was performed by comparing trace of WT SK4 channel currents in the absence and presence of 20 μM BA6b9. Whole-cell SK4 K⁺ currents were activated using a voltage ramp protocol from −100 mV to +60 mV for 150 milliseconds. As presented in FIG. 4C, showing that BA6b9 inhibits SK4 currents by 56% at 20 μM. These data indicate that the exemplary compound BA6b9 is a more potent SK4 channel inhibitor compared to BA6B (see, FIG. 3B).

To further examine the inhibition of BA6b9, the Ca²⁺-dependent activation of WT SK4 channels was measured in inside-out macro-patches in the absence and presence of 10 μM BA6b9. FIG. 8C shows BA6b9 inhibited SK4 currents by 66% in inside-out macro-patches.

To conclude, the obtained data indicate that the halide-bearing exemplary compounds BA40 and BA100 activated the SK4 channel, while the mono-N-alkylated exemplary compounds BA6B and BA6b9 inhibited the SK4 channel. Di-N-alkylated exemplary compounds such as BA6A were inactive as SK4 channel modulators.

### Example 3

### Molecular Docking and Experimental Validation

In order to study the inhibiting activity of the exemplary compound BA6b9, molecular docking to the Ca²⁺-bound state I (6CNN) of the SK4 channel was simulated in the presence of bound PIP2. PIP2 was docked as described hereinabove, and the obtained image is shown in FIG. 5A. The docking pose of BA6b9 indicated that compound BA6b9 fits into a gorge formed by the boundaries of S1 and S4 transmembrane helices and the S4-S5 linker in close proximity to the bound PIP2, where the inward-facing heptane tail contacts the bottom of the BA6b9-binding pocket, which is formed by the CaM linker. In the S4-S5 linker, the guanidinium group of the R191 residue interacts via H-bonding with the carbonyl oxygen of the benzoxazolyl group of BA6b9 (distance H—O=1.9 Å), as can be seen in FIG. 5B. Residue H192 could engage into aromatic H-bonding or π-π stacking interactions between the imidazole moiety of histidine and the benzoxazole ring of BA6b9 (distance=4.2 Å). In the CaM linker, BA6b9 clashes with the residue of M76 while it could be involved in hydrophobic interactions with M72, as presented in FIG. 5B.

In pursue to compare the inhibitory activity of the exemplary benzoxazolone BA6b9 to that of the benzimidazolone activators 1-EBIO and the exemplary compound BA40, molecular docking to the SK4 channel (6CNN) by was performed and presented in FIG. 5C. The docking indicates that the compounds that act as channel openers (1-EBIO and BA40) dock similarly to one another, but differently from BA6b9; Unlike BA6b9, 1-EBIO and BA40 are distant from the CaM residue M76 (distance >11 Å) and thereby do not clash with it. Both molecules sit at the bottom of the inward-facing phosphate head groups of PIP2, suggesting stabilization of the SK4 channel-CaM complex by interacting with the CaM linker loop (residues R74 and K75) between the two lobes, as presented in FIG. 5C, left.

To examine ex-vivo the interactions between BA6b9 and the simulated residues seen in FIG. 5B, residues that are in atomic proximity and are expected to interact with BA6b9 were mutated. Such mutated residues were, for example, R191 and H192 of linker S4-S5, M72 and M76 of the CaM linker as well as L19, E22, Y179, S181, A184, Q187 and R189, which are more distant from BA6b9.

The obtained data is shown in FIG. 6A. As can be seen, mutations Y179S, S181A, A184R, Q187A, and R189A, which are of residues in linker S4-S5 and are fairly remote from the docked ligand, exhibited inhibition by 20 μM BA6b9 comparable to that of WT SK4 channels (ranging from 59% to 46% inhibition). The mutants L19W and E22A of residues located at the N-terminus in atomic vicinity with but theoretically do not interact with BA6b9, showed 46% and 56% inhibition, respectively, similar to that obtained with WT (56%).

However, as can also be seen in FIG. 6A, mutations at residues at atomic vicinity and interaction with BA6b9, such as R191A and H192 Å, displayed decreased inhibition of 30% and 16%, respectively, compared to WT.

The residues Arg191 and His192 are specific to SK4 channels, and are replaced respectively by Asparagine and Threonine in SK1, SK2 and SK3 channels, as shown in FIG. 7A.

To examine the hypothesized role of residues R191 and H192 in the selective inhibition of SK4 channel by BA6b9, the effect of BA6b9 on the SK1-SK3 homomeric channels was examined.

As can be seen in FIG. 7B, the presence of the exemplary compound BA6b9 (20 μM), did not lead to modulation of WT SK1 channel currents. In addition, as FIG. 7C depicts, a minimal affect was observed in each of SK1, SK2 and SK3 channels with a drug-to-control current ratio of 1.18, 1.34, and 0.95, respectively.

In the CaM linker, which forms the floor of the BA6b9 binding pocket, the mutants M72A and M76A exhibited minor or decreased inhibition of 0% and 19%, respectively, as is shown in FIG. 8A and FIG. 8B, indicating that the exemplary compound targets this domain of CaM.

The mutant residue R352Q of helix B, which is distant from BA6b9 and is unable to be activated by PIP4,5-kinase, as indicated in FIGS. 2A and 6A, showed decreased inhibition (31%) compared to WT, suggesting an allosteric impact of helix B site to the docking stability of the ligand. Thus, it is assumed that BA6b9 inhibits the SK4 channel by preventing the calcified CaM N-lobe to properly contact its S4-S5 linker site to open the channel, which is expected to affect the Ca²⁺-dependence of SK4 channels.

To further examine the postulated allosteric inhibition of the exemplary compound BA6b9, the Ca²⁺-dependent activation of WT SK4 channels was measured in inside-out macro-patches in the absence and presence of 10 μM BA6b9. As can be seen in FIG. 4A, BA6b9 lowers the sensitivity of SK4 channels to Ca²⁺ by about 7-fold with an EC₅₀ for Ca²⁺ of 435 nM. In 1 μM free-Ca²⁺, 10 μM BA6b9 inhibited SK4 currents by 66% in inside-out macro-patches, as presented in FIG. 8C.

To conclude, the BA6b9 molecule was designed as described in Example 2. Simulation of its docking suggests H-bonding and π-π stacking interactions with the calmodulin-PIP2 binding domain (CPBD), a region located at a boundary of the channel proximal C-terminus and the S4-S5 linker. BA6b9 mainly interacts with the residues of R191 and H192, which are not conserved in SK1-SK3 subunits, therefore conferring SK4 channel selectivity. The clash of BA6b9 onto the CaM linker region results in decreased inhibition, as displayed by mutants M72A and M76A. The right-shift of the Ca²⁺-dependence for SK4 channel activation suggests that BA6b9 allosterically inhibits SK4 channels. By acting at the calmodulin-PIP2 interface, it is suggested that BA6b9 disrupts the interaction of the calcified CaM N-lobe with the proximal S₄₅A helix, thus preventing SK4 channel gate opening.

### Example 4

### Effects of BA6b9 on Isolated Heart Preparations

As discussed in the Background section, previous studies suggested that SK4 channel blockers may serve as an interesting therapeutic approach for the treatment of cardiac arrhythmias. Therefore, the effect of BA6b9 on heart cells was examined in animal models. To examine the impact of BA6b9 on cardiac electrophysiology, the Langendorff method of perfused isolated rat and guinea pig hearts was used. The obtained data are presented in FIGS. 9A-E and 10A-H, respectively.

In adult healthy rat heart, 10 μM of the exemplary compound BA6b9 prolonged both AERP (from 56 millisecond (ms) to 63 ms) and AVERP (from 84 ms to 89 ms), but did not change the VERP, as seen in FIG. 9A. BA6b9 also produced bradycardia by decreasing heart rate (from 228 bpm to 196 bpm; FIG. 9B) and increasing PR interval (from 56 ms to 68 ms; FIG. 9C).

To quantify AF induction, the AF induction score (AFIS) system was used, wherein higher threshold receives lower score (see: Materials and Experimental Methods). BA6b9 (10 μM) reduced the AFIS by 48%, from a score of 3.1 (0.3 μM carbachol alone) to 1.6, as can be seen in FIG. 9D.

Sustained AF was induced using a burst pacing protocol in which pacing intensity is gradually increased from 1.5× diastolic threshold to 6× diastolic threshold. While 100% AF induction was obtained with carbachol (0.3 μM) alone, 70% induction was achieved when the exemplary compound BA6b9 was present, as can be seen in FIG. 9E.

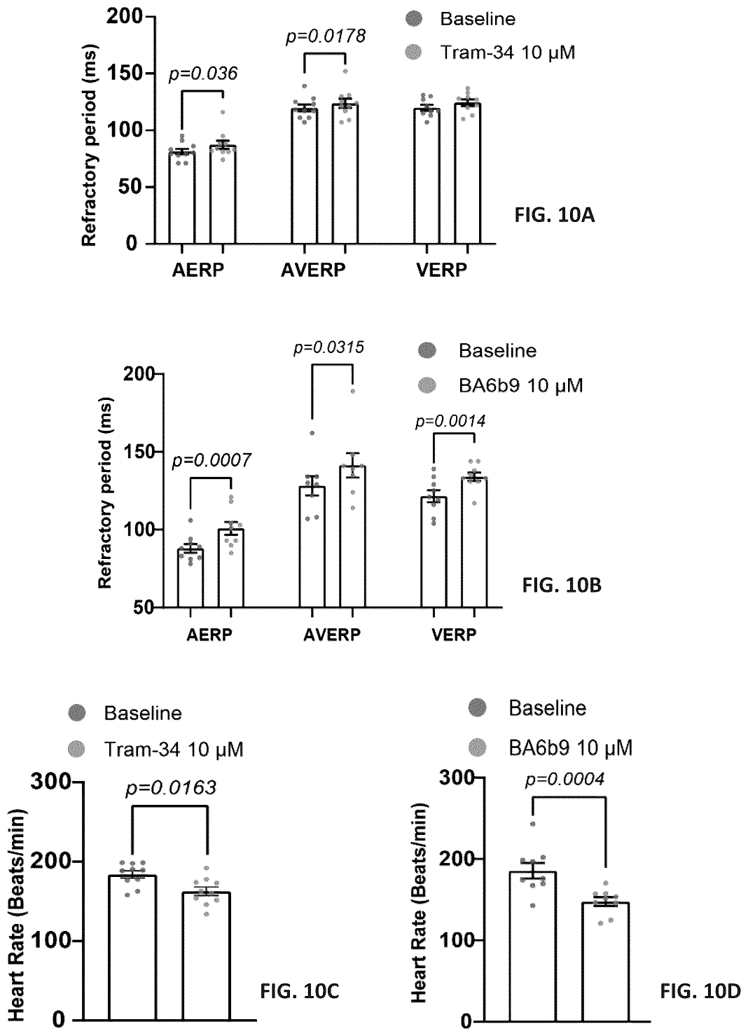

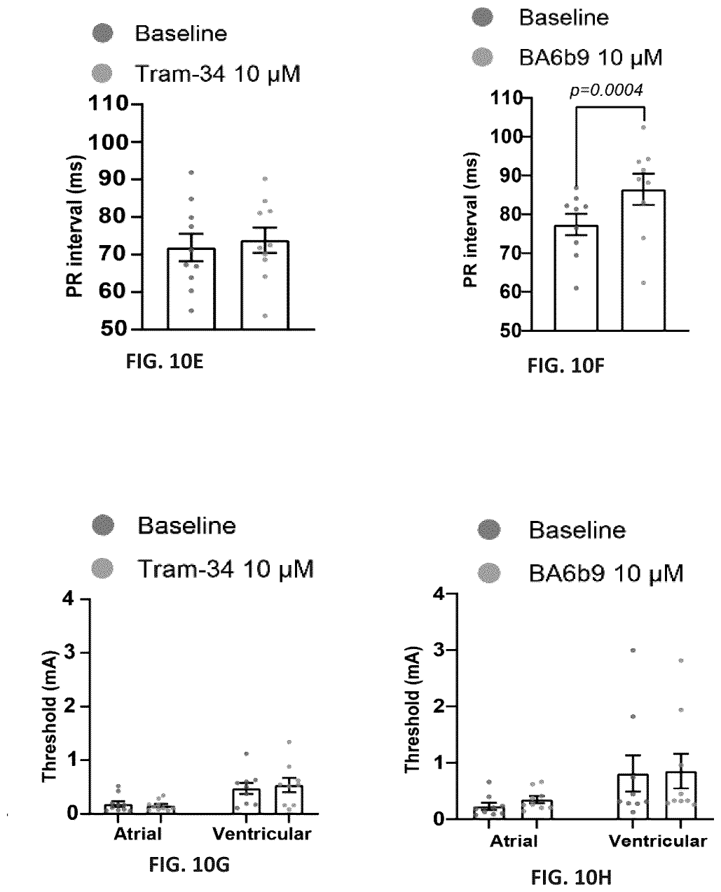

Similar electrophysiological trends were obtained in isolated adult healthy guinea pig hearts. When compared to the currently known SK4 inhibitor Tram-34, the exemplary compound BA6b9 showed similar or improved performances in refractory period measurements as seen in FIG. 10A and FIG. 10B, decreased heart rate as seen in FIG. 10C and FIG. 10D and increasing PR interval as seen in FIG. 10E and FIG. 10F. Neither Tram-34 nor BA6b9 affected the action potential activation current (threshold), as seen in FIG. 10G and FIG. 10H.

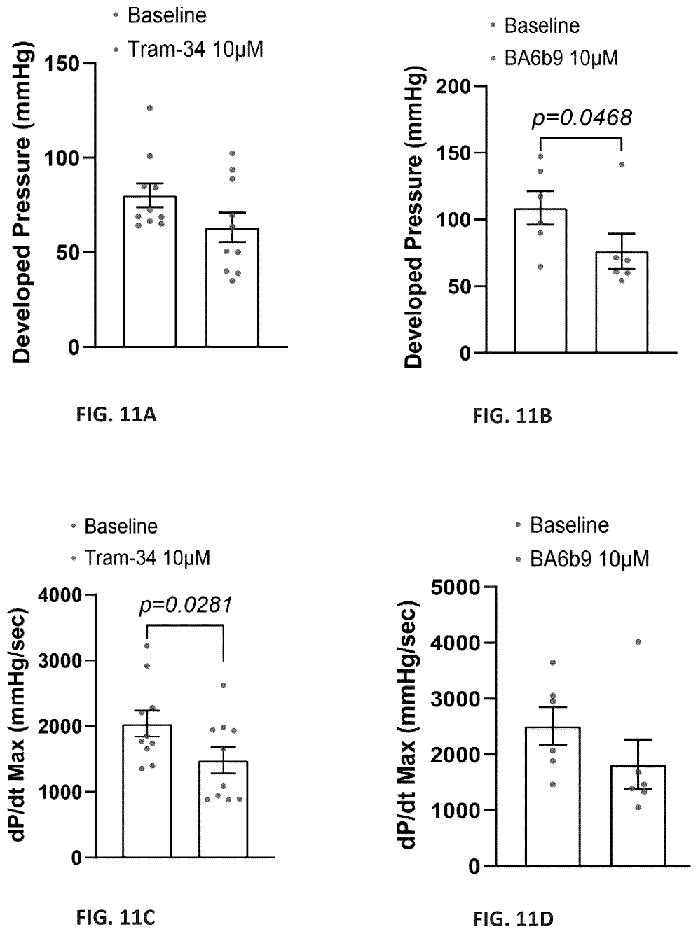

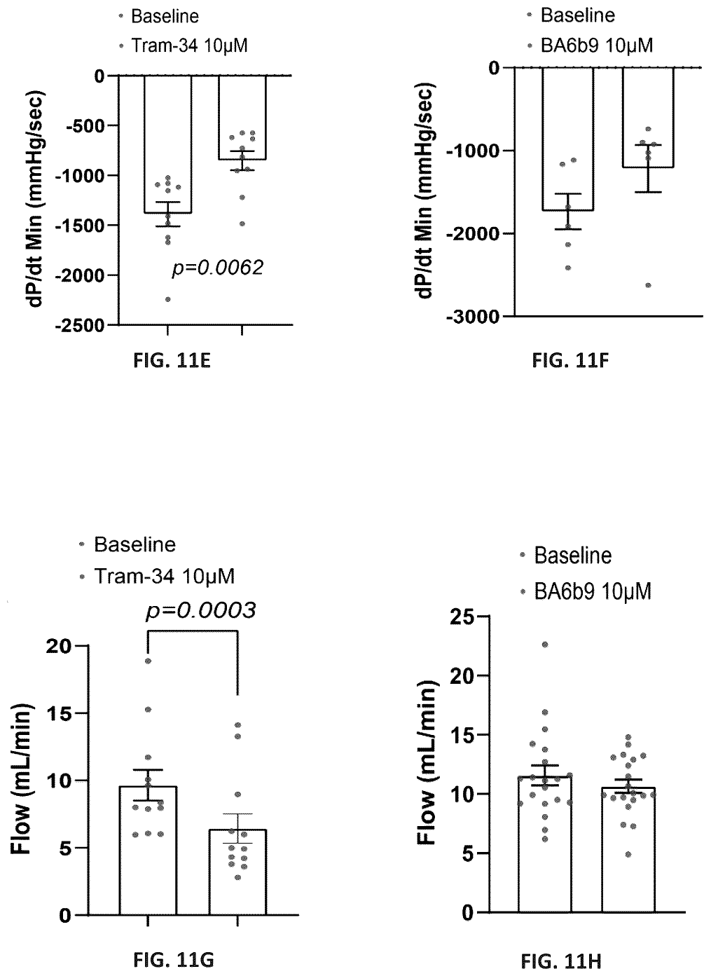

The hemodynamic parameters were tested in rat hearts, while comparing Tram-34 and the exemplary compound BA6b9. While Tram-34 did not affect the left ventricular filling pressure (developed pressure), BA6b9 reduced it, as can be seen in FIG. 11A and FIG. 11B, respectively. FIGS. 11C and 11D, and FIGS. 11E and 11F depicts the pressure change in left ventricular contractility, indicating Tram-34 reduces both parameters while BA6b9 does not effect it.

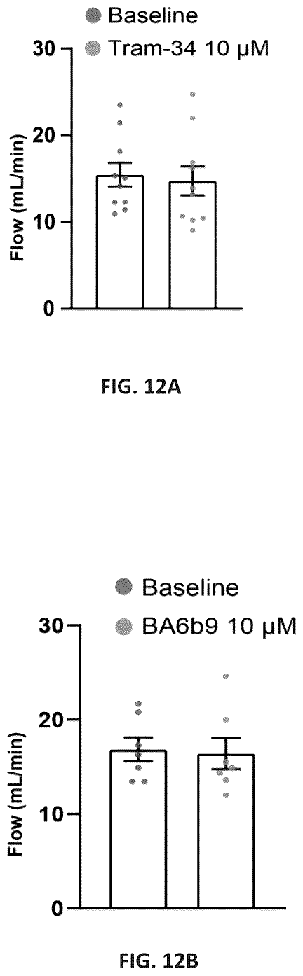

While Tram-34 significantly lowered the coronary flow, the exemplary compound BA6b9 had minimal effect on it, as can be seen in FIG. 11G and FIG. 11H, respectively. In guinea pig hearts, both Tram-34 and BA6b9 had no effect on the coronary flow as can be seen in FIG. 12A and FIG. 12B, respectively.

In conclusion, as VERP remains unchanged, the results show that an exemplary compound according to some of the present embodiments, BA6b9, prolongs AERP and AVERP, decreased heart rate and increases PR interval in adult healthy rat and guinea-pig isolated hearts, and therefore may be useful for anti-arrhythmic therapy.

### Example 5

### Effects of BA6b9 on Atrial Fibrillation Substrate and Structural Remodeling Post-Myocardial Infarction

In order to evaluate the effect of the exemplary compound BA6b9 on myocardial infarction as a model cardiac arrhythmia, rats were examined in-vivo post-myocardial infarction (MI) as described herein.

Following electrode implantation and animal recovery, baseline EP measurements were performed followed by random division of the animals to treatment with the exemplary compound vs. treatment with vehicle.

The in-vivo data for EF, AERF and AF substrate parameters are presented in FIGS. 13A-D.

The data indicate that for rats with heart failure (HF) one week post-myocardial infarction (MI), subsequent treatment of 2 weeks daily administration of BA6b9 (20 mg/kg) does not affect EF, as can be seen in FIGS. 13A and 13B, but markedly attenuates atrial fibrosis (AF) induction and duration, as illustrated in FIGS. 13C and 13D. The effect of the exemplary compound BA6b9 in-vivo was further assessed by examining the structural remodeling post-MI in left atrial (LA) cells. FIGS. 14A-D present the histochemical analysis and quantifications of % LA fibrosis and smooth muscle actin (SMA) following the post-MI treatment.

As can be seen, data show a reduction both in LA fibrosis and in LA SMA following treatment with the exemplary compound, BA6b9. Without being bound to any particular theory, it is assumed that due to the important role of SK4 channels in the activation of macrophages and fibroblasts, inhibition of the SK4 channel by the exemplary compound results in their downregulation and thus in reduced fibrosis. To conclude, the exemplary compound according to some of the present embodiments, BA6b9, ameliorates AF substrate and structural remodeling post-MI, and thus substantiate its use in the treatment of cardiac arrhythmia, and specifically in the treatment of MI and of cardiac fibrosis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of downregulating an activity of an SK4 channel in a subject in need thereof, comprising administering to said subject a compound represented by Formula I: 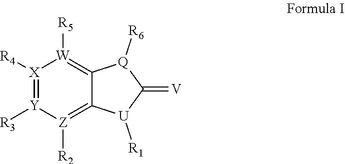
or a pharmaceutically acceptable salt thereof,
wherein:
X, Y, Z and W are each independently carbon or nitrogen, wherein when Z is nitrogen R₂ is absent; when Y is nitrogen, R₃ is absent; when X is nitrogen, R₄ is absent and when W is nitrogen, R₅ is absent;
Q, and U are each independently selected from O, S and N, wherein when Q is O or S, R₆ is absent; and when U is O or S, R₁ is absent; at least one of Q and U being nitrogen (N);
V is O, S or NR₇;
R₁, R₆ and R₇, when present, are each independently selected from hydrogen, alkyl, alkenyl, alkynyl, cycloalkyl, heteroalicyclic, aryl, heteroaryl, hydroxy, alkoxy, thiol, carboxylate, thiocarboxylate, thiohydroxy, carbonyl, carbamate, provided that one of R₁ and R₆ is an alkyl of at least 5 carbon atoms in length; and that when Q and U are each nitrogen, only one of R₁ and R₆ is the alkyl of at least 5 carbon atoms in length; and
R₂, R₃, R₄ and R₅ are each independently selected from hydrogen, alkyl, alkenyl, alkynyl, aryl, heteroaryl, halo, hydroxy, thiol, alkoxy, aryloxy, amine, amide, azo, azide, cyano, carbamyl, hydrazine, ether, ester, carbonyl, sulfonyl, sulfinyl, sulfonamide, thionyl, thioalkoxy, thioalkyl, thioaryl, thioester, thiocarbamide, thiocarbamate.

2. The method of claim 1, wherein V is O.

3. The method of claim 1, wherein U is N and R₁ is the alkyl of at least 5 carbon atoms in length.

4. The method of claim 3, wherein Q is N, and R₆ is hydrogen or an alkyl of up to 3 carbon atoms in length.

5. The method of claim 3, wherein Q is O.

6. The method of claim 1, wherein R₂, R₃, R₄ and R₅ are each hydrogen.

7. The method of claim 1, wherein R₂, R₃, R₄ and R₅ are each independently selected from hydrogen, alkyl, halo, nitro, cyano, alkoxy and thioalkoxy.

8. The method of claim 1, wherein X, Y, W and Z are each carbon.

9. The method of claim 1, wherein:
X, Y, W and Z are each carbon;
R₂, R₃, R₄ and R₅ are each hydrogen; and
V is O.

10. The method of claim 9, wherein:
U is N;
R₁ is the alkyl of at least 5 carbon atoms in length;
Q is N; and
R₆ is hydrogen or an alkyl of up to 3 carbon atoms in length.

11. The method of claim 9, wherein:
U is N;
R₁ is the alkyl of at least 5 carbon atoms in length; and
Q is O.

12. A method of downregulating an activity of an SK4 channel in a subject in need thereof, the method comprising administering to the subject a compound capable of interacting with the calmodulin-PIP2 binding domain of the SK4 channel to thereby interfere with the Ca²⁺-dependent activation of the SK4 channel, the compound being represented by Formula I: 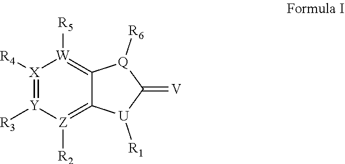
or a pharmaceutically acceptable salt thereof,
wherein:
X, Y, Z and W are each independently carbon or nitrogen, wherein when Z is nitrogen R₂ is absent; when Y is nitrogen, R₃ is absent; when X is nitrogen, R₄ is absent and when W is nitrogen, R₅ is absent;
Q, and U are each independently selected from O, S and N, wherein when Q is O or S, R₆ is absent; and when U is O or S, R₁ is absent; at least one of Q and U being nitrogen (N);
V is O, S or NR₇;
R₁, R₆ and R₇, when present, are each independently selected from hydrogen, alkyl, alkenyl, alkynyl, cycloalkyl, heteroalicyclic, aryl, heteroaryl, hydroxy, alkoxy, thiol, carboxylate, thiocarboxylate, thiohydroxy, carbonyl, carbamate, provided that one of R₁ and R₆ is an alkyl of at least 5 carbon atoms in length; and that when Q and U are each nitrogen, only one of R₁ and R₆ is the alkyl of at least 5 carbon atoms in length; and
R₂, R₃, R₄ and R₅ are each independently selected from hydrogen, alkyl, alkenyl, alkynyl, aryl, heteroaryl, halo, hydroxy, thiol, alkoxy, aryloxy, amine, amide, azo, azide, cyano, carbamyl, hydrazine, ether, ester, carbonyl, sulfonyl, sulfinyl, sulfonamide, thionyl, thioalkoxy, thioalkyl, thioaryl, thioester, thiocarbamide, thiocarbamate.
